SEQUENCE LISTING (13 sequences)

SEQ ID NO: 1 - protein, 12 aa, synthetic construct
  REGION 1..12: note = Artificial peptide sequence
GGGPQGIWGQGK

SEQ ID NO: 2 - protein, 12 aa, synthetic construct
  REGION 1..12: note = Artificial peptide sequence
GGGIQQWGPGGK

SEQ ID NO: 3 - protein, 12 aa, synthetic construct
  REGION 1..12: note = Artificial peptide sequence
GGGGGIPQQWGK

SEQ ID NO: 4 - protein, 4 aa, synthetic construct
  REGION 1..4: note = Cell adhesive peptide sequence
RGDS

SEQ ID NO: 5 - protein, 4 aa, synthetic construct
  REGION 1..4: note = Cell adhesive peptide sequence
RDGS

SEQ ID NO: 6 - protein, 4 aa, synthetic construct
  REGION 1..4: note = Cell adhesive peptide sequence
RGES

SEQ ID NO: 7 - protein, 4 aa, synthetic construct
  REGION 1..4: note = Cell adhesive peptide sequence
REGS

SEQ ID NO: 8 - protein, 5 aa, synthetic construct
  REGION 1..5: note = Cell adhesive peptide sequence
IKVAV

SEQ ID NO: 9 - protein, 5 aa, synthetic construct
  REGION 1..5: note = Cell adhesive peptide sequence
VVIAK

SEQ ID NO: 10 - protein, 5 aa, synthetic construct
  REGION 1..5: note = Cell adhesive peptide sequence
YIGSR

SEQ ID NO: 11 - protein, 5 aa, synthetic construct
  REGION 1..5: note = Cell adhesive peptide sequence
YSRIG

SEQ ID NO: 12 - protein, 4 aa, synthetic construct
  REGION 1..4: note = DGEA peptide sequence
DGEA

SEQ ID NO: 13 - protein, 4 aa, synthetic construct
  REGION 1..4: note = Cell adhesive peptide sequence
DAEG

(19) United States
(12) Patent Application Publication
(10) Pub. No.: US 2024/0335500 A1
(43) Pub. Date: Oct. 10, 2024

(54) CONTROLLING PRO-INFLAMMATORY MACROPHAGE PHENOTYPE THROUGH BIOFUNCTIONAL HYDROGEL DESIGN

(71) Applicant: UNIVERSITY OF FLORIDA RESEARCH FOUNDATION, INCORPORATED, Gainesville, FL (US)

(72) Inventors: Erika MOORE, Gainesville, FL (US); Aakanksha JHA, Gainesville, FL (US)

(21) Appl. No.: 18/290,734

(22) PCT Filed: Jul. 11, 2022
(86) PCT No.: PCT/US2022/073593

§ 371 Date: Jan. 19, 2024

Related U.S. Application Data
(60) Provisional application No. 63/225,177, filed on Jul. 23, 2021.

(51) Int. Cl. A61K 38/07; A61K 47/60; C07K 5/113; C08L 71/02

(52) U.S. Cl. CPC A61K 38/07; A61K 47/60; C07K 5/1021; C08L 71/02; C08L 2203/02

(57) ABSTRACT

Described herein are hydrogel compositions comprising DGEA for use in treating diseases or disorders associated with excessive or sustained inflammation. Also described herein are compositions comprising DGEA for use in inhibiting activation of pro-inflammatory M1 macrophages.

### CROSS-REFERENCE TO RELATED APPLICATIONS

This application claims the benefit of and priority to U.S. Provisional Patent Application No. 63/225,177, filed on Jul. 23, 2021, the contents of which are incorporated by reference herein in their entirety for all purposes.

### REFERENCE TO A SEQUENCE LISTING

The official copy of the sequence listing is submitted electronically via Patent Center as an XML formatted sequence listing with a file named 580787SEQLIST created on Jul. 11, 2022, and having a size of 17,175 bytes and is filed concurrently with the specification. The sequence listing contained in this XML formatted document is part of the specification and is herein incorporated by reference in its entirety.

### FIELD

The presently disclosed subject matter relates generally to compositions comprising DGEA for the treatment of inflammatory diseases or disorders.

### BACKGROUND

Inflammation is the body's defense mechanism in response to injurious stimuli, such as damaged cells or pathogens (L. Chen et al., “Inflammatory responses and inflammation-associated diseases in organs,” Oncotarget. 2018). Inflammation initiates wound healing as the first stage of the immune response. In situations where inflammation persists, a healthy wound healing cascade may transition to a chronic inflammatory disorder. Throughout the world, 3 out of 5 people die due to chronic inflammatory conditions, such as cardiovascular and pulmonary diseases, osteoarthritis, and diabetes (R. Pahwa and I. Jialal, “Chronic Inflammation—StatPearls—NCBI Bookshelf,” Stat Pearls. 2019). Thus, there is a dire need to control inflammation through manipulation of the immune response. The subject matter described herein addresses this unmet need.

### BRIEF SUMMARY

In one aspect, the presently disclosed subject matter is directed to a crosslinked poly(alkylene glycol)-based hydrogel composition, comprising:
a cell adhesive peptide covalently conjugated with a first poly(alkylene glycol);
a cleavable peptide linker covalently conjugated with a second and third poly(alkylene glycol); and
DGEA covalently conjugated with a fourth poly(alkylene glycol);
wherein, said first, second, third, and fourth poly(alkylene glycol), in each instance, are the same or different.

In another aspect, the presently disclosed subject matter is directed to a method of treating a disease or disorder associated with excessive or sustained inflammation in a subject in need thereof, comprising administering to said subject a therapeutically effective amount of a composition comprising DGEA.

In another aspect, the presently disclosed subject matter is directed to a method of inhibiting activation of pro-inflammatory M1 macrophages, comprising contacting one or more macrophages with a composition comprising DGEA.

In another aspect, the presently disclosed subject matter is directed to a method of inhibiting activation of pro-inflammatory M1 macrophages in a subject in need thereof, comprising administering to the subject a therapeutically effective amount of a composition comprising DGEA.

These and other aspects are described herein.

### BRIEF DESCRIPTION OF THE DRAWINGS

FIG. 1 shows the soluble delivery of DGEA to 2D cultures. In the inset (a), M0 macrophages were polarized to the M1 phenotype by adding 100 ng/mL LPS and 10 ng/mL IFNG (M1 media). The control condition had no soluble DGEA added to the macrophage medium, while the experimental condition had 5 mM soluble DGEA dissolved in the macrophage medium. In the inset (b), immunofluorescent images of cells are shown, which were stained for DAPI and iNOS with (left) no soluble DGEA and (right) with 5 mM soluble DGEA. In the inset (c), is a graph representing the number of iNOS+ cells normalized to DAPI+ cells in control conditions (n=7) and in samples with 5 mM DGEA (n=8). Data represent a student's t-test in both conditions. Analysis was performed on ImageJ and GraphPad Prism. Data are statistically significant (p<0.05).

FIG. 2 shows soluble delivery of DGEA in a 3D matrix of PEG hydrogels. Inset (a) shows a representation of dissolved 5 mM DGEA peptide in control hydrogels incorporating 3.5 mM PEG-RGDS and 5% PEG-PQ-PEG. Raw 264.7 cells encapsulated within 5 μL droplet of control hydrogels. Inset (b) shows iNOS and DAPI stained images of M1 macrophages with and without the addition of 5 mM soluble DGEA. Inset (c) shows iNOS+ cells that were normalized to DAPI+ cells (n=4). Data represent a student's t-test in both conditions. Analysis was performed on ImageJ and GraphicPad Prism. Data are nonsignificant to each other (p>0.05).

FIG. 3 is directed to the immobilization of DGEA in a PEG hydrogel and assessing its conjugation efficiency. Inset (a) shows the chemical structure of PEG-DGEA after crosslinking Acryl-PEG-SVA with DGEA. Inset (b) shows a representation of the formation of experimental PEG-DGEA hydrogel with the cell-adhesive PEG-RGDS peptide and enzyme-cleavable PEG-PQ-PEG peptide. Inset (c) shows a MALDI-ToF analysis of PEG peptide only (top right) and PEG-DGEA to assess conjugation of PEG with peptide.

FIG. 4. Inset (a) shows a schematic of how M0 macrophages were polarized to the M1 phenotype by adding 100 ng/mL LPS and 10 ng/mL IFNγ. Raw 264.7 cells were encapsulated within PEG-RGDS (control) and PEG-RGDS+PEG-DGEA (experimental) hydrogels to test the inhibitory behavior of DGEA on M1 macrophage activation. Inset (b) shows immunofluorescent images of samples stained for DAPI and iNOS. M1 macrophages encapsulated in (left) control hydrogels (3.5 mM RGDS, 5% PQ), and (right) PEG-DGEA experimental hydrogels (5 mM DGEA, 3.5 mM RGDS, 5% PQ). Inset (c) shows the results of a student's t-test, which was used to access the number of iNOS+ cells/DAPI+ cells in both conditions (n=5). Significance is depicted by (p<0.05). Inset (d) shows an ELISA analysis of TNFα expression in the conditioned media of M1 macrophages encapsulated in PEG-DGEA hydrogels (**p<0.01).

FIG. 5 is directed to a series of experiments in which macrophages derived from a healthy donor were encapsulated in control and experimental hydrogels for analysis. Inset (a) shows immunofluorescent images of samples stained for DAPI and iNOS. M1 macrophages encapsulated in (left) control hydrogels (3.5 mM RGDS, 5% PQ), and (right) PEG-DGEA experimental hydrogels (5 mM DGEA, 3.5 mM RGDS, 5% PQ). Inset (b) shows the results of a student's t-test, which was used to assess the number of iNOS+ cells/DAPI+ cells under both conditions (n=8). Significance is depicted by (p<0.05). Inset (c) shows a process for deriving macrophages from a healthy donor.

FIG. 6 shows a timeline of experimental design for modes of DGEA assessment on M1 macrophage phenotype.

FIG. 7 shows bar graphs of the results from a series of RT-PCR experiments. The results validate the inflammation inhibiting properties of DGEA by looking at pro-inflammatory gene expressions. The genes assessed include TNFα (a); IL-6 (b); and iNOS (c). As shown by the bar graphs, the DGEA peptide significantly reduces expression of pro-inflammatory mediators (i.e. inflammation signaling molecules such as iNOS and inflammatory cytokines such as TNFα).

FIG. 8 shows bar graphs displaying the results of iNOS expression in (a) M0, (b) M1 and (C) M2 encapsulated raw 264.7 macrophages in control and PEG-DGEA hydrogels, and the CD206 expression in (d) (M0), (e) (M1), and (f) (M2) encapsulated raw 264.7 macrophages in control and PEG-DGEA hydrogels. As shown in the graphs, DGEA reduced iNOS expression in M0, M1 and M2 macrophages. Meanwhile, CD206 expression in M0 macrophages encapsulated in PEG-DGEA was higher than in control hydrogels.

FIG. 9 shows bar graphs of the results of iNOS and CD206 expression in M1 and M2 encapsulated raw 264.7 macrophages in PEG-DGEA hydrogels. As shown by the graphs, DGEA did not appear to have any significant effects on CD206 expression in M1 or M2 macrophage phenotypes.

FIG. 10 shows bar graphs of the results of iNOS and CD206 expression in M1 and M2 encapsulated raw 264.7 macrophages in PEG-DGEA hydrogels. DGEA demonstrated inhibition of M1 macrophages. Both iNOS+ and CD206+ cells were higher in M2 macrophages than M1. After performing a student's t-tests on the data, CD206+ cells were significantly higher in M2 macrophages. iNOS expression demonstrated a similar trend in M2 macrophages.

FIG. 11 shows immunofluorescent images of samples stained for DAPI and iNOS.

FIG. 12 shows immunofluorescent images of samples stained for DAPI and CD206.

### DETAILED DESCRIPTION

The subject matter described herein relates to hydrogel compositions comprising DGEA for use in treating diseases or disorders associated with excessive or sustained inflammation. In certain other embodiments, the subject matter described herein is directed to hydrogel compositions comprising DGEA for use in inhibiting activation of pro-inflammatory M1 macrophages.

Macrophages are immune cells integral to the promotion of wound healing and resolution of inflammation (C. J. Ferrante and S. J. Leibovich, “Regulation of Macrophage Polarization and Wound Healing,” Adv. Wound Care, 2012). Macrophages are highly plastic cells that change their functions based on environmental cues, thereby acquiring various phenotypes. This process is also known as macrophage polarization (F. O. Martinez and S. Gordon, “The M1 and M2 paradigm of macrophage activation: Time for reassessment,” F1000Prime Rep., 2014). Unstimulated macrophages are typically termed M0 macrophages. M1 macrophages, also called “classically activated”, are associated with a pro-inflammatory phenotype (C. Atri, F. Z. Guerfali, D. Laouini, “Role of human macrophage polarization in inflammation during infectious diseases,” Int. J. Mol. Sci. 19, 1801 (2018)). They express pro-inflammatory cytokines such as tumor necrosis factor (TNF)-α and interferon (IFN)-γ (G. A. Duque and A. Descoteaux, “Macrophage cytokines: Involvement in immunity and infectious diseases,” Frontiers in Immunology. 2014; M. Rath, I. Müller, P. Kropf, E. I. Closs, and M. Munder, “Metabolism via arginase or nitric oxide synthase: Two competing arginine pathways in macrophages,” Frontiers in Immunology, vol. 5, no. OCT. Frontiers Media S. A., 2014). To promote inflammation, M1 macrophages express inducible nitric oxide synthase (iNOS) which has antimicrobial effects towards pathogens. iNOS is a hallmark marker for M1 macrophages as its upregulation is induced by a hypoxic environment, pro-inflammatory cytokines (IFNγ, TNFα) as well as microbial factors such as lipopolysaccharide (LPS) (M. Rath, et al. Frontiers in Immunology, vol. 5, no. OCT. Frontiers Media S. A., 2014). M2, or “alternatively activated” macrophages, are an inflammation-resolution phenotype that express anti-inflammatory cytokines. When macrophages are exposed to inflammatory stimuli during the wound healing cascade, they release cytokines that initiate inflammation. A disproportionate production of inflammatory cytokines causes an excess of the M1 macrophages (M. Rath, et al. Frontiers in Immunology, vol. 5, no. OCT. Frontiers Media S. A., 2014; L. Parisi et al., “Macrophage Polarization in Chronic Inflammatory Diseases: Killers or Builders?” Journal of Immunology Research. 2018). The dysregulation of the M1 macrophage population density at a site of inflammation or wound healing can contribute to chronic inflammation, and promote chronic inflammatory disorders (L. Parisi et al., Journal of Immunology Research. 2018; P. Krzyszczyk, R. Schloss, A. Palmer, and F. Berthiaume, “The role of macrophages in acute and chronic wound healing and interventions to promote pro-wound healing phenotypes,” Frontiers in Physiology, vol. 9, no. MAY. Frontiers Media S. A., p. 419, 1 May 2018). Interventions which therapeutically repolarize macrophages, in the form of preventing the M1 phenotype, for example, can be beneficial for treatment of chronic inflammatory diseases.

Extracellular matrix (ECM) proteins also inform macrophage polarization. Collagen along with other ECM proteins such as fibrin or laminin have often been used as biomaterials to assess cell function. To offer manipulation of the microenvironments, ECM-derived peptides, such as RGDS found in fibronectin, collagen IV-derived GFPGER, and laminin derived IKVAV, are utilized to alter cellular responses via integrin receptor interactions (S. Vigier and T. Fülöp, “Exploring the Extracellular Matrix to Create Biomaterials,” in Composition and Function of the Extracellular Matrix in the Human Body, InTech, 2016). Specifically, the α2β1 integrin receptor interacts with Type I collagen and mediates extracellular signals to macrophages (W. D. Staatz, K. F. Fok, M. M. Zutter, S. P. Adams, B. A. Rodriguez, and S. A. Santoro, “Identification of a tetrapeptide recognition sequence for the α2β1 integrin in collagen,” J Biol. Chem., 1991; C. Popov et al., “Integrins α2β1 and α11β1 regulate the survival of mesenchymal stem cells on collagen i,” Cell Death Dis., vol. 2, no. 7, July 2011; M. Mizuno, R. Fujisawa, and Y. Kuboki, “Type I collagen-induced osteoblastic differentiation of bone-marrow cells mediated by collagen-α2β1 integrin interaction,” J. Cell. Physiol., 2000; Y. Liu and T. Segura, “Biomaterials-Mediated Regulation of Macrophage Cell Fate,” Frontiers in Bioengineering and Biotechnology. 2020). Integrin α2β1 appears to play a pivotal role in macrophage polarization by affecting downstream signaling pathways (B. H. Cha et al., “Integrin-Mediated Interactions Control Macrophage Polarization in 3D Hydrogels,” Adv. Healthc. Mater., 2017). A study by Cha et al. showed that binding via integrin α2β1 strongly increased CD86 (M1 marker) expression and reduced CD206 (M2 marker) expression in gelatin methacryloyl (GelMA) and poly (ethylene glycol) diacrylate (PEGDA) hydrogels. Whereas, blocking the binding sites of α2β1 via DGEA-coated plates led to a higher expression of CD206 (M2).

With a focus on the function of α2β1 in macrophage activation, the role of DGEA was analyzed in macrophage manipulation via ECM-peptide interactions. DGEA is a tetrapeptide of the sequence Asp-Gly-Glu-Ala, which corresponds to residues 435-438 of the Type I collagen sequence (W. D. Staatz, K. F. Fok, M. M. Zutter, S. P. Adams, B. A. Rodriguez, S. A. Santoro, “Identification of a tetrapeptide recognition sequence for the α2β1 integrin in collagen.” J Biol. Chem. 266, 7363-7367 (1991)). Staatz et al. synthesized peptides, 12-13 amino acids in length, to assess inhibition of platelet adhesion to collagen. Particularly, the α2β1 integrin receptor was chosen due to the overlap between collagen and laminin recognition. After assessing the importance of aspartic acid and glutamic acid in adhesion of cells, it was concluded that DGEA is the minimal tetrapeptide recognition sequence for α2β1. In a study by Mizuno et al., collagen-integrin interactions were interrupted by addition of the DGEA peptide to the culture (M. Mizuno, R. Fujisawa, and Y. Kuboki, “Type I collagen-induced osteoblastic differentiation of bone-marrow cells mediated by collagen-α2β1 integrin interaction,” J. Cell. Physiol., 2000). Fishman et al. have also demonstrated DGEA to be effective in blocking adhesion to collagen via α2β1 integrin mediation (D. A. Fishman et al., “Metastatic dissemination of human ovarian epithelial carcinoma is promoted by α2β1-integrin-mediated interaction with type I collagen,” Invasion Metastasis 18(1), 15-26 (1998)). As shown herein, DGEA can block the binding sites of the α2β1 integrin. It was therefore hypothesized that the presence of DGEA can reduce the M1 macrophage phenotype, suggesting DGEA as a potential inhibitor for M1 macrophage polarization.

To mitigate the induction of pro-inflammatory M1 macrophages, the macrophage response to DGEA both in a soluble form and in an immobilized hydrogel were assessed using polyethylene glycol (PEG) conjugated with DGEA. A previous study by Wu et al. demonstrated immobilization of DGEA in a hydrogel to investigate adhesion of valve interstitial cells via integrins (Y. Wu, K. J. Grande-Allen, J. L. West, “Adhesive peptide sequences regulate valve interstitial cell adhesion, phenotype and extracellular matrix deposition,” Cell. Mol. Bioeng. 9(4), 479 (2016)). Other studies have utilized PEG-DGEA to better understand effects of shear and tension in tenocytes, cells of the tendon, and to model tissue-specific regeneration of endothelial cells and keratocytes (D. Patel, S. Sharma, S. J. Bryant, “Tenocyte attachment to collagen mimetic peptides increase mechano-sensitivity to shear and tension,” Orthop. Proc. 97, 11 (2018); J. Reddy, M. Gerasimov, M. Griffth, “Functional modification of collagen like peptides for cellular specificity and function. Investig,” Ophthalmol. Vis. Sci. 60, 9 (2019)). The results indicate that DGEA can reduce M1 activation via both soluble delivery in the media and in the immobilized form. As shown herein, DGEA, a collagen-derived peptide, can influence macrophage phenotype. The methods and hydrogel design described herein can be used to manipulate pro-inflammatory macrophage activation, as well as be employed as a biomaterial tool to address chronic inflammatory diseases.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literatures, patents, and similar materials differs from or contradicts this application, including but not limited to defined terms, term usage, described techniques, or the like, this application controls.

### I. Definitions

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.1% of the specified amount.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

As used herein, “ECM” refers to extracellular matrix.

As used herein, the term “peptide” refers to linear or cyclic or branched compounds containing amino acids, amino acid equivalents or other non-amino groups, while still retaining the desired functional activity of a peptide. Peptide equivalents can differ from conventional peptides by the replacement of one or more amino acids with related organic acids such as p-aminobenzoic acid (PABA), amino acid analogs, or the substitution or modification of side chains or functional groups. Peptide equivalents encompass peptide mimetics or peptidomimetics, which are organic molecules that retain similar peptide chain pharmacophore groups as are present in the corresponding peptide. The term “peptide” refers to peptide equivalents as well as peptides.

As used herein, a “culture” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

An “effective amount” or “therapeutically effective amount” of a compound or composition is that amount of compound or composition which is sufficient to provide a beneficial effect to the subject to which the compound is administered. An “effective amount” of a delivery vehicle is that amount sufficient to effectively bind or deliver a compound or composition.

The terms “patient,” “subject,” “individual,” and the like are used interchangeably herein, and refer to any animal, or cells thereof whether in vitro or in situ, amenable to the methods described herein. In certain nonlimiting embodiments, the patient, subject or individual is a mammal, and in other embodiments, the mammal is a human.

The term “treating” or “treatment” means to stabilize or improve the clinical symptoms of the subject. In another embodiment, “treating” or “treatment” also means to relieve or alleviate at least one symptom associated with such condition, or to slow or reverse the progression or anticipated progression of such condition, at bringing about ameliorations of the symptoms of the conditions described herein.

As used herein, the terms “prevent,” “prevention,” “inhibit” or “inhibiting” refer to stopping, hindering, suppressing and/or slowing down the onset of developing adverse effects and at least symptom associated with medical condition described herein.

Additional definitions are provided below.

I. Crosslinked Poly(alkylene glycol)-based Hydrogel Compositions

In one aspect, the subject matter described herein is directed to a crosslinked poly(alkylene glycol)-based hydrogel composition, comprising:
a cell adhesive peptide covalently conjugated with a first poly(alkylene glycol);
a cleavable peptide linker covalently conjugated with a second and third poly(alkylene glycol); and
DGEA (SEQ ID NO: 12) covalently conjugated with a fourth poly(alkylene glycol);
wherein, said first, second, third, and fourth poly(alkylene glycol), in each instance, are the same or different.

As used herein, a “cell adhesive peptide” is a peptide that promotes cell adhesion. Cell adhesion peptides can bind to the cell membrane and trigger adhesion of cells.

In certain embodiments of the crosslinked poly(alkylene glycol)-based hydrogel composition, the cell adhesive peptide in the cell adhesive peptide covalently conjugated with the first poly(alkylene glycol) is selected from the group consisting of RGDS (SEQ ID NO: 4), RDGS (SEQ ID NO: 5), RGES (SEQ ID NO: 6), REGS (SEQ ID NO: 7), IKVAV (SEQ ID NO: 8), VVIAK (SEQ ID NO: 9), YIGSR (SEQ ID NO: 10), YSRIG (SEQ ID NO: 11), DAEG (SEQ ID NO: 13), and combinations thereof.

In certain embodiments of the crosslinked poly(alkylene glycol)-based hydrogel composition, the cell adhesive peptide covalently conjugated with a first poly(alkylene glycol) is present in the crosslinked poly(alkylene glycol)-based hydrogel composition at a concentration of about 0.5 mM to about 10 mM. In certain embodiments of the crosslinked poly(alkylene glycol)-based hydrogel composition, the cell adhesive peptide covalently conjugated with a first poly(alkylene glycol) is present in the crosslinked poly(alkylene glycol)-based hydrogel composition at a concentration of about 0.5 mM, 1.0 mM, 1.5 mM, 2.0 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 5.0 mM, 5.5 mM, 6.0 mM, 6.5 mM, 7.0 mM, 8.0 mM, 9.0 mM, or 10 mM.

As used herein, a “cleavable peptide linker” refers to a bioconjugation linker that can connect two or more molecules together and then can be cleaved once exposed to either enzyme, photo-irradiation, or chemical reagent.

In certain embodiments of the crosslinked poly(alkylene glycol)-based hydrogel composition, the cleavable peptide linker in the cleavable peptide linker covalently conjugated with a second and third poly(alkylene glycol) is selected from the group consisting of GGGPQGIWGQGK (SEQ ID NO: 1), GGGIQQWGPGGK (SEQ ID NO: 2), and GGGGGIPQQWGK (SEQ ID NO: 3).

In certain embodiments of the crosslinked poly(alkylene glycol)-based hydrogel composition, the cleavable peptide linker covalently conjugated with a second and third poly(alkylene glycol) is present in the crosslinked poly(alkylene glycol)-based hydrogel at about 2% to about 15%. In certain embodiments of the crosslinked poly(alkylene glycol)-based hydrogel composition, the cleavable peptide linker covalently conjugated with a second and third poly(alkylene glycol) is present in the crosslinked poly(alkylene glycol)-based hydrogel at about 2 wt %, 2.5 wt %, 3 wt %, 3.5 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.5 wt %, 7 wt %, 7.5 wt %, 8 wt %, 8.5 wt %, 9 wt %, 10 wt %, 11 wt %, 12 wt %, 13 wt %, 14 wt %, or 15 wt %.

As used herein, the “DGEA covalently conjugated with a fourth poly(alkylene glycol)” in the crosslinked poly(alkylene glycol)-based hydrogel composition refers to a poly(alkylene glycol)-conjugate of a tetrapeptide of the sequence Asp-Gly-Glu-Ala (SEQ ID NO: 12).

In certain embodiments of the crosslinked poly(alkylene glycol)-based hydrogel composition, the DGEA (SEQ ID NO: 12) covalently conjugated with the fourth poly(alkylene glycol) is present in the crosslinked poly(alkylene glycol)-based hydrogel composition at a concentration of about 1 mM to about 15 mM. In certain embodiments of the crosslinked poly(alkylene glycol)-based hydrogel composition, the DGEA covalently conjugated with the fourth poly(alkylene glycol) is present in the crosslinked poly(alkylene glycol)-based hydrogel composition at a concentration of about 1 mM, 1.5 mM, 2 mM, 2.5 mM, 3 mM, 3.5 mM, 4 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6 mM, 6.5 mM, 7 mM, 7.5 mM, 8 mM, 8.5 mM, 9 mM, 10 mM, 11 mM, 12 mM, 13 mM, 14 mM, or 15 mM.

Any suitable poly(alkylene glycol) (PAG), such as a poly(propylene glycol), poly(butylene glycol), or poly(ethylene glycol) (PEG), can be used for the first, second, third, and fourth poly(alkylene glycol). In certain embodiments, each of the first, second, third, and fourth poly(alkylene glycol) (PAG) is a poly(ethylene glycol) (PEG) and the crosslinked poly(alkylene glycol)-based hydrogel is a crosslinked poly(ethylene glycol)-based hydrogel. In certain embodiments, the PAG is an acrylated poly(alkylene glycol), such as an acrylated poly(ethylene glycol). Unless indicated otherwise, the term “acrylate” refers to a compound or polymer conjugate containing an acrylate functional group, CH₂═CHCOOR, wherein R is the polymer or molecule (i.e. peptide). In certain embodiments, the peptide conjugates disclosed herein contain two acrylate groups, such as the acrylate-PEG cleavable peptide linker conjugate comprising a first and second acrylate-PEG group.

In certain embodiments of the crosslinked poly(alkylene glycol)-based hydrogel composition, the first, second, third, and fourth poly(alkylene glycol), in each instance, is selected from the group consisting of acrylate-PEG-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succnimidyl succinate (PEG-SS), and acrylate-PEG-maleimide (PEG-MAL).

One terminal end of the poly(alkylene glycol) used to prepare the cell adhesive peptide conjugate and/or the DGEA conjugate can be functionalized with an acrylate group while another terminal end may be functionalized with a group such as valerate, N-hydroxylsuccinimide, succinimidyl carboxymethyl ester, succinimidyl amido succinate, succinimidyl carbonate, succinimidyl succinate, succinimidyl carbonate, succinimidyl glutarate, or maleimide which is capable of reacting with a functional group (for example, an amino group —NH₂) on a peptide (to form a polymer-peptide macromer or acrylate-PEG cell adhesive peptide conjugate). In certain embodiments, two poly(alkylene glycol)s (the second and third PAG as referred to herein) are used for the preparation of the cleavable peptide linker conjugate.

In certain embodiments of the crosslinked poly(alkylene glycol)-based hydrogel composition, wherein the conjugate of each of the cell adhesive peptide covalently conjugated with the first poly(alkylene glycol), said cleavable peptide linker covalently conjugated with the second and third poly(alkylene glycol), and the DGEA covalently conjugated with the fourth poly(alkylene glycol), comprises:
an acrylate-PEG cell adhesive peptide conjugate;
an acrylate-PEG cleavable peptide linker conjugate comprising a first and second acrylate-PEG group, wherein the cleavable peptide linker is disposed between the first and second acrylate-PEG group; and
an acrylate-PEG DGEA conjugate.

The PAG for use in the synthesis of the crosslinked poly(alkylene glycol)-based hydrogel compositions can have a number average molecular weight in the range of 2 to 20 kDa and can be dissolved in an aqueous buffered saline at a concentration range of 2-20% weight/volume for use in the formation of the cell adhesive peptide, cleavable peptide linker, and DGEA conjugates.

As used herein, “crosslinked poly(alkylene glycol)-based hydrogel composition” refers to a hydrogel poly(alkylene glycol) that has undergone crosslinking.

Photopolymerization (crosslinking) the conjugate polymer-peptide macromers with an optional co-monomer can proceed in the presence of an ultraviolet (UV) photoinitiator. A suitable co-monomer includes, but is not limited to, n-vinyl pyrrolidone (NVP). Other suitable co-monomers include other types of ethylenically unsaturated compounds containing at least one carbon-carbon double bond capable of participating in a photopolymerization involving ethylenically unsaturated functional groups, such as acrylate groups, on the peptide conjugate macromers disclosed herein. Such carbon-carbon double bonds may be present in the co-monomer in the form of vinyl or acrylate groups, for example.

In certain embodiments of the crosslinked poly(alkylene glycol)-based hydrogel composition: the cell adhesive peptide in the cell adhesive peptide covalently conjugated with the first poly(alkylene glycol) is RGDS (SEQ ID NO: 4), wherein the cell adhesive peptide covalently conjugated with the first poly(alkylene glycol) is present in the hydrogel composition at a concentration of about 3.5 mM; the cleavable peptide linker in the cleavable peptide linker covalently conjugated with the second and third poly(alkylene glycol) is GGGPQGIWGQGK (SEQ ID NO: 1), wherein the cleavable peptide linker covalently conjugated with the second and third poly(alkylene glycol) is present in the hydrogel composition at about 5 wt %; the DGEA covalently conjugated with the fourth poly(alkylene glycol) is present in the hydrogel composition at a concentration of about 5 mM; and wherein, the first, second, third, and fourth poly(alkylene glycol), in each instance, is the same; wherein the poly(alkylene glycol) is a poly(ethylene glycol), and wherein the poly(ethylene glycol) is acrylate-PEG-succinimidyl valerate (PEG-SVA).

In certain embodiments of the crosslinked poly(alkylene glycol)-based hydrogel composition: the conjugate of each of the cell adhesive peptide covalently conjugated with the first poly(alkylene glycol), the cleavable peptide linker covalently conjugated with the second and third poly(alkylene glycol), and the DGEA (SEQ ID NO: 12) covalently conjugated with the fourth poly(alkylene glycol), comprises:
an acrylate-PEG cell adhesive peptide conjugate, wherein the acrylate-PEG cell adhesive peptide conjugate is present in the crosslinked poly(alkylene glycol)-based hydrogel composition at a concentration of about 0.5 mM to about 10 mM;
an acrylate-PEG cleavable peptide linker conjugate comprising a first and second acrylate-PEG group, wherein the cleavable peptide linker is disposed between the first and second acrylate-PEG group; wherein the acrylate-PEG cleavable peptide linker conjugate comprising a first and second acrylate-PEG group is present in the crosslinked poly(alkylene glycol)-based hydrogel composition at a concentration of about 2 wt % to about 15 wt %; and
an acrylate-PEG DGEA conjugate, wherein the acrylate-PEG DGEA conjugate is present in the crosslinked poly(alkylene glycol)-based hydrogel composition at a concentration of about 1 mM to about 15 mM.

In certain embodiments of the crosslinked poly(alkylene glycol)-based hydrogel composition, the cell adhesive peptide in the acrylate-PEG cell adhesive peptide conjugate is RGDS (SEQ ID NO: 4). In certain embodiments of the crosslinked poly(alkylene glycol)-based hydrogel composition, the cleavable peptide linker in the acrylate-PEG cleavable peptide linker conjugate comprising the first and second acrylate-PEG group is GGGPQGIWGQGK (SEQ ID NO: 1).

In certain embodiments of the crosslinked poly(alkylene glycol)-based hydrogel composition, the acrylate-PEG cell adhesive peptide conjugate is present in the crosslinked poly(alkylene glycol)-based hydrogel composition at a concentration of about 3.5 mM. In certain embodiments of the crosslinked poly(alkylene glycol)-based hydrogel composition, the acrylate-PEG cleavable peptide linker conjugate comprising the first and second acrylate-PEG group is present in the crosslinked poly(alkylene glycol)-based hydrogel composition at a concentration of about 5 wt %. In certain embodiments of the crosslinked poly(alkylene glycol)-based hydrogel composition, the acrylate-PEG DGEA conjugate is present in the crosslinked poly(alkylene glycol)-based hydrogel composition at a concentration of about 5 mM.

Hydrogels can generally absorb a great deal of fluid and, at equilibrium, typically are composed of 60-90% fluid and only 10-30% polymer. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. “Preparation and Characterization of Cross-linked Hydrophilic Networks in Absorbent Polymer Technology,” Brannon-Peppas and Harland, Eds. 1990, Elsevier: Amsterdam, pp 45-66; Peppas and Mikos. “Preparation Methods and Structure of Hydrogels in Hydrogels in Medicine and Pharmacy,” Peppas, Ed. 1986, CRC Press: Boca Raton, Fla., pp 1-27). Methods for preparing the crosslinked poly(alkylene glycol)-based hydrogel composition are described in the examples herein.

### III. Methods of Treatment

In certain embodiments, the subject matter described herein is directed to a method of treating a disease or disorder associated with excessive or sustained inflammation in a subject in need thereof, comprising administering to the subject a therapeutically effective amount of a composition comprising DGEA.

In certain embodiments, the subject matter described herein is directed to a method of inhibiting activation of pro-inflammatory M1 macrophages, comprising contacting one or more macrophages with a composition comprising DGEA. In certain embodiments, the macrophages to be contacted are unactivated (M0) macrophages.

In certain other embodiments, the subject matter described herein is directed to a method of inhibiting activation of pro-inflammatory M1 macrophages in a subject in need thereof, comprising administering to the subject a therapeutically effective amount of a composition comprising DGEA.

In certain embodiments of the method of treating a disease or disorder associated with excessive or sustained inflammation in a subject in need thereof, of the method of inhibiting activation of pro-inflammatory M1 macrophages, or of the method of inhibiting activation of pro-inflammatory M1 macrophages in a subject in need thereof, the composition comprising DGEA is a hydrogel composition comprising DGEA and one or more synthetic polymers. In certain embodiments, the one or more synthetic polymers are selected from the group consisting of acrylate-PEG-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succnimidyl succinate (PEG-SS), and acrylate-PEG-maleimide (PEG-MAL). In certain embodiments, the composition comprising DGEA is a hydrogel composition comprising DGEA and one or more synthetic polymers is a crosslinked poly(alkylene glycol)-based hydrogel composition, comprising:
a cell adhesive peptide covalently conjugated with a first poly(alkylene glycol);
a cleavable peptide linker covalently conjugated with a second and third poly(alkylene glycol); and
DGEA covalently conjugated with a fourth poly(alkylene glycol);
wherein, said first, second, third, and fourth poly(alkylene glycol), in each instance, are the same or different.

### A. Treating Inflammation

Inflammation normally is a localized, protective response to trauma or microbial invasion that destroys, dilutes, or walls-off the injurious agent and the injured tissue. It is characterized in the acute form by the classic signs of pain, heat, redness, swelling, and loss of function. Microscopically, it involves a complex series of events, including dilation of arterioles, capillaries, and venules, with increased permeability and blood flow, exudation of fluids, including plasma proteins, and leukocyte migration into the area of inflammation.

Diseases characterized by inflammation are significant causes of morbidity and mortality in humans. Commonly, inflammation occurs as a defensive response to invasion of the host by foreign, particularly microbial, material. Responses to mechanical trauma, toxins, and neoplasia also may results in inflammatory reactions. The accumulation and subsequent activation of leukocytes are central events in the pathogenesis of most forms of inflammation. Deficiencies of inflammation compromise the host. Excessive inflammation caused by abnormal recognition of host tissue as foreign or prolongation of the inflammatory process may lead to inflammatory diseases as diverse as diabetes, arteriosclerosis, cataracts, reperfusion injury, and cancer, to post-infectious syndromes such as in infectious meningitis, rheumatic fever, and to rheumatic diseases such as systemic lupus erythematosus and rheumatoid arthritis. The centrality of the inflammatory response in these varied disease processes makes its regulation a major element in the prevention control or cure of human disease.

Methods are described herein for decreasing inflammation in a subject. In certain embodiments, the methods are for treating a disease or disorder associated with excessive or sustained inflammation. The subject can have inflammation of any organ, including organs of the digestive system, skin, nervous system, lymph system, cardiovascular system, or endocrine system. In some examples, inflammation of the joints or skin can be treated using the presently described methods. The inflammation can be acute or chronic. The subject can be any subject of interest, including healthy or immunocompromised subjects. Methods are also described herein for reducing an inflammatory response in vitro, such as in cultures of isolated animal cells. The subject can be a human or a veterinary subject.

As used herein, the term “chronic inflammation” refers to prolonged and persistent inflammation marked chiefly by new connective tissue formation; it may be a continuation of an acute form or a prolonged low-grade form. In one embodiment, “chronic inflammation” refers to “excessive or sustained inflammation.”

In one embodiment, as used herein, the term “excessive or sustained inflammation” refers to ongoing inflammatory responses that have gone beyond the homeostatic condition of providing the host with immune defense mechanisms adequate for protection against an acute infection or injury. In another embodiment, as used herein, the term “excessive or sustained inflammation” refers to ongoing inflammatory responses that is causing an unacceptable level of tissue damage as a result of the response and is not related to protection against an acute infection or injury.

In some embodiments, the subject is suffering from a disease or physiological condition, such as inflamed joints or muscles. In certain embodiments, the subject suffers from inflammation of mucosal surfaces, such as canker sores, or hemorrhoids. In certain embodiments, the subject suffers from a disease or condition of the skin, such as acne, psoriasis, herpes sores, or allergic reactions, including reactions to poison ivy, and insect bites. In some embodiments, the subject has an allergy, asthma, arthritis or colitis. In some embodiments, the subject is suffering from an acute inflammatory condition, such an acute allergic reaction or sprains, bruises and muscle damage from sports-injury or accidents, or sunburn. In one example, the subject is suffering from inflammation related to the natural progression of the menstrual cycle, but leading to excessive pain and cramps. In specific embodiments, the subject suffers from any inflammatory diseases and conditions. In some embodiments, the subject has an allergy, asthma, atherosclerosis, dermatitis (such as allergic chronic contact dermatitis and environmental chronic contact dermatitis), laminitis, reactive airway diseases and processes (such chronic obstructive pulmonary disease (“COPD”), inflammatory airway disease (“IAD”), inflammatory bowel disease, and rheumatoid arthritis, ulcerative colitis, Crohn's disease, stroke-induced brain cell death, traumatic brain injury, ankylosing spondylitis, fibromyalgia. Autoimmune diseases that include inflammation, such as multiple sclerosis, systemic lupus erythematosus, scleroderma, systemic sclerosis, and Sjögren's syndrome can also be treated using the methods disclosed herein. In certain embodiments, the subject has an inflammatory disease or disorder selected from the group consisting of arthritis, asthma, inflammatory bowel disease, chronic obstructive pulmonary disease (COPD), allergic rhinitis, vasculitis, inflammatory neuropathy, psoriasis, systemic lupus erythematosis (SLE), chronic thyroiditis, Hashimoto's thyroiditis, Addison's disease, polymyalgia rheumatica, Sjögren's syndrome, and Churg-Strauss syndrome.

Methods are also disclosed herein for treating or preventing inflammatory lung disease in a subject. Inflammatory lung diseases include, but are not limited to pneumonia, ARDS, respiratory distress of prematurity, chronic bronchitis, chronic obstructive pulmonary disease (COPD), cystic fibrosis, pulmonary fibrosis, and pulmonary sarcoidosis. The method includes administering a therapeutically effective amount of the composition comprising DGEA, to a subject having or at risk of developing inflammatory lung disease, thereby treating or preventing the inflammatory lung disease. In one embodiment, the composition comprising DGEA can be administered locally, such as by inhalation. In another embodiment, the composition comprising DGEA is administered systemically, such as by intravenous injection.

Local administration of the composition comprising DGEA is performed by methods well known to those skilled in the art. By way of example, one method of administration to the lungs of an individual is by inhalation through the use of a nebulizer or inhaler. For example, the composition comprising DGEA is formulated in an aerosol or particulate and drawn into the lungs using a standard nebulizer well known to those skilled in the art.

Methods are also disclosed herein for the treatment of arthritis. Arthritis is an inflammatory disease that affects the synovial membranes of one or more joints in the body, is the most common type of joint disease. Billions of dollars are spent annually for the treatment of arthritis and for lost days of work associated with the disease. The disease is usually oligoarticular (affects few joints), but may be generalized. The joints commonly involved include the hips, knees, lower lumbar and cervical vertebrae, proximal and distal interphangeal joints of the fingers, first carpometacarpal joints, and first tarsometatarsal joints of the feet.

Rheumatoid Arthritis (RA) is a chronic, systemic, inflammatory disease that affects the synovial membranes of multiple joints. RA considered an acquired autoimmune disease, and genetic factors appear to play a role in its development. In most cases of RA, the subject has remissions and exacerbations of the symptoms. Rarely does the disease resolve completely, although at times the symptoms might temporarily remit.

A method is disclosed herein for treating or preventing an inflammatory arthropathy in a subject. The method includes administering a therapeutically effective amount of the composition comprising DGEA, to a subject having or at risk of developing an inflammatory arthropathy, such as arthritis, thereby treating or preventing the inflammatory arthropathy. In one embodiment, the composition comprising DGEA can be administered locally, such as by intra-articular injection. In another embodiment, the composition comprising DGEA can be administered systemically. In other embodiments, the composition comprising DGEA can be administered intravenously, such as, for example, through an intravenous implant.

Local administration of the composition comprising DGEA for the treatment of arthritis is performed by methods well known to those skilled in the art. By way of example, one method of administration to the knee, hip and/or shoulder of an individual is by intra-articular injection. For administration to the knee, for example, the joint to be injected is washed with a betadine solution or other antiseptic. A solution of an anesthetic, such as about one percent lidocaine hydrochloride is injected into the skin and subcutaneous tissue. A 3-way stopcock/needle assembly is utilized to administer the compound via an 18-30 gauge needle. The composition comprising DGEA is injected into the joint space using a standard lateral approach well known to those skilled in the art. The needle and needle tract are cleansed by flushing with 1% lidocaine hydrochloride through the 3-way stopcock assembly as the needle is withdrawn. The knee is then moved through a flexion-extension arc and then immobilized in full extension. The patient is then confined to bed for approximately 24 hours to minimize movement and minimize leakage of the anti-inflammatory fraction from the joint.

Regardless of how the composition comprising DGEA is provided or administered, the methods disclosed herein can result in a transient relief from acute or chronic inflammation, such as a reduction in pain, redness, itching, or swelling. Reduction of inflammation can be determined by measuring the changes in a subject's temperature (systemic or local), redness, blood flow, swelling, or other ways to physically measure effects of inflammation. Also, reduction in inflammation can be measured by taking blood samples for evaluation of inflammatory markers in serum, plasma, or cells. For example, C-reactive protein, one or more cytokines, prostaglandins, or lipid peroxidation status can be measured, and a reduction in the above markers will indicate a reduction in inflammation. Reduction of inflammation may also be recorded by questionnaires pertaining to pain, itching, or other subjective measures related to inflammation. Compositions comprising the described compositions comprising DGEA including compositions comprising one or more pharmaceutically acceptable carriers are thus provided for both local (such as topical or inhalational) and/or systemic (such as oral or intravenous) use to treat the various inflammatory conditions descried herein. Therefore, the disclosure includes within its scope pharmaceutical compositions comprising the composition comprising DGEA formulated for use in human or veterinary medicine. While the composition comprising DGEA will typically be used to treat human subjects, it may also be used to treat similar or identical diseases in other vertebrates, such as other primates, dogs, cats, horses, and cows. A suitable administration format may best be determined by a medical practitioner for each subject individually. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42: 2S, 1988. The dosage form of the pharmaceutical composition will be determined by the mode of administration chosen.

In certain embodiments, a therapeutically effective amount of the composition comprising DGEA is formulated for administration to the skin. Formulations suitable for topical administration can include dusting powders, ointments, cremes, gels or sprays for the administration of the active compound to cells, such as skin cells. Such formulations may optionally include an inorganic pigment, organic pigment, inorganic powder, organic powder, hydrocarbon, silicone, ester, triglyceride, lanolin, wax, cere, animal or vegetable oil, surfactant, polyhydric alcohol, sugar, vitamin, amino acid, antioxidant, free radical scavenger, ultraviolet light blocker, sunscreen agents, preservative, fragrance, thickener, or combinations thereof.

In certain embodiments, the composition comprising DGEA can be used in cosmetic formulations (e.g., skincare cream, sunscreen, decorative make-up products, and other dermatological compositions) in various pharmaceutical dosage forms, and especially in the form of oil-in-water or water-in-oil emulsions, solutions, gels, or vesicular dispersions. The cosmetic formulations may take the form of a cream which can be applied either to the face or to the scalp and hair, as well as to the human body, in particular those portions of the body that are chronically exposed to sun.

In some cosmetic formulations, additives can be included such as, for example, preservatives, bactericides, perfumes, antifoams, dyes, pigments which have a coloring action, surfactants, thickeners, suspending agents, fillers, moisturizers, humectants, fats, oils, waxes or other customary constituents of a cosmetic formulation, such as alcohols, polyols, polymers, foam stabilizers, electrolytes, organic solvents, or silicone derivatives.

Cosmetic formulations typically include a lipid phase and often an aqueous phase. The lipid phase can be chosen from the following group of substances: mineral oils, mineral waxes, such as triglycerides of capric or of caprylic acid, castor oil; fats, waxes and other natural and synthetic fatty substances, esters of fatty acids with alcohols of low C number, for example with isopropanol, propylene glycol or glycerol, or esters of fatty alcohols with alkanoic acids of low C number or with fatty acids; alkyl benzoates; silicone oils, such as dimethylpolysiloxanes, diethylpolysiloxanes, diphenylpolysiloxanes and mixed forms thereof.

If appropriate, the aqueous phase of the formulations according to the present disclosure include alcohols, diols or polyols of low C number and ethers thereof, such as ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols of low C number, for example ethanol, isopropanol, 1,2-propanediol and glycerol, and, in particular, one or more thickeners, such as silicon dioxide, aluminum silicates, polysaccharides and derivatives thereof, for example hyaluronic acid, xanthan gum and hydroxypropylmethylcellulose, or poly-acrylates.

An exemplary cosmetic formulation is as an additive to a sunscreen composition as a lotion, spray or gel, for administration to the skin to prevent or treat inflammation. A sunscreen can additionally include at least one further UVA filter and/or at least one further UVB filter and/or at least one inorganic pigment, such as an inorganic micropigment. The UVB filters can be oil-soluble or water-soluble. Oil-soluble UVB filter substances can include, for example: 3-benzylidenecamphor derivatives, such as 3-(4-methylbenzylidene)camphor and 3-benzylidenecamphor; 4-aminobenzoic acid derivatives, such as 2-ethylhexyl 4-(dimethylamino)benzoate and amyl 4-(dimethylamino)benzoate; esters of cinnamic acid, such as 2-ethylhexyl 4-methoxycinnamate and isopentyl 4-methoxycinnamate; derivatives of benzophenone, such as 2-hydroxy-4-methoxybenzophenone, 2-hydroxy-4-methoxy-4′-methylbenzophenone and 2,2′-dihydroxy-4-methoxybenzophenone; esters of benzalmalonic acid, such as di(2-ethylhexyl)4-methoxybenzalmalonate. Water-soluble UVB filter substances can include the following: salts of 2-phenylbenzimidazole-5-sulphonic acid, such as its sodium, potassium or its triethanolammonium salt, and the sulphonic acid itself; sulphonic acid derivatives of benzophenones, such as 2-hydroxy-4-methoxybenzophenone-5-sulphonic acid and salts thereof; sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)benzenesulphonic acid and salts thereof. The list of further UVB filters mentioned which can be used in combination with the active agent(s) according to the disclosure is not intended to be limiting.

For treatment of the skin, a therapeutically effective amount of the composition comprising DGEA also can be locally administered to only an affected area of the skin, such as in the form of an ointment. In one embodiment, the ointment is an entirely homogenous semi-solid external agent with a firmness appropriate for easy application to the skin. Such an ointment can include fats, fatty oils, lanoline, Vaseline, paraffin, wax, hard ointments, resins, plastics, glycols, higher alcohols, glycerol, water or emulsifier and a suspending agent. Using these ingredients as a base, a decoy compound can be evenly mixed. Depending on the base, the mixture can be in the form of an oleaginous ointment, an emulsified ointment, or a water-soluble ointment oleaginous ointments use bases such as plant and animal oils and fats, wax, Vaseline and liquid paraffin. Emulsified ointments are comprised of an oleaginous substance and water, emulsified with an emulsifier. They can take either an oil-in-water form (O/W) or a water-in-oil-form (W/O). The oil-in-water form (O/W) can be a hydrophilic ointment. The water-in-oil form (W/O) initially lacks an aqueous phase and can include hydrophilic Vaseline and purified lanoline, or it can contain a water-absorption ointment (including an aqueous phase) and hydrated lanoline. A water-soluble ointment can contain a completely water-soluble Macrogol base as its main ingredient.

Pharmaceutically acceptable carriers include a petroleum jelly, such as VASELINE®, wherein the petroleum jelly contains 5% stearyl alcohol, or petroleum jelly alone, or petroleum jelly containing liquid paraffin. Such carriers enable pharmaceutical compositions to be prescribed in forms appropriate for consumption, such as tablets, pills, sugar-coated agents, capsules, liquid preparations, gels, ointments, syrups, slurries, and suspensions. When locally administered into cells in an affected area or a tissue of interest, the composition comprising DGEA can be administered in a composition that contains a synthetic or natural hydrophilic polymer as the carrier. Examples of such polymers include hydroxypropyl cellulose and polyethylene glycol. The composition comprising DGEA can be mixed with a hydrophilic polymer in an appropriate solvent. The solvent is then removed by methods such as air-drying, and the remainder is then shaped into a desired form (for example, a sheet) and applied to the target site. Formulations containing such hydrophilic polymers keep well as they have a low water-content. At the time of use, they absorb water, becoming gels that also store well. In the case of sheets, the firmness can be adjusted by mixing a polyhydric alcohol with a hydrophilic polymer similar to those above, such as cellulose, starch and its derivatives, or synthetic polymeric compounds. Hydrophilic sheets thus formed can be used. A therapeutically effective amount of the composition comprising DGEA can also be incorporated into bandages.

In certain embodiments, the composition comprising DGEA can be formulated for administration by inhalation, such as, but not limited to, formulations for the treatment of asthma. Inhalational preparations include aerosols, particulates, and the like. In general, the goal for particle size for inhalation is about 1 μm or less in order that the pharmaceutical reach the alveolar region of the lung for absorption. However, the particle size can be modified to adjust the region of disposition in the lung. Thus, larger particles can be utilized (such as about 1 to about 5 μm in diameter) to achieve deposition in the respiratory bronchioles and air spaces. In addition, oral formulations may be liquid (e.g., syrups, solutions, or suspensions), or solid (e.g., powders, pills, tablets, or capsules).

For administration by inhalation, the composition comprising DGEA can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges for use in an inhaler or insufflator can be formulated containing a powder mix of the compound and a suitable powder base such as lactose or starch.

The compositions or pharmaceutical compositions also can be administered by any route, including parenteral administration, for example, intravenous, intraperitoneal, intramuscular, intraperitoneal, intrasternal, or intraarticular injection or infusion, or by sublingual, oral, topical, intranasal, or transmucosal administration, or by pulmonary inhalation. When the composition comprising DGEA is provided as parenteral compositions, e.g. for injection or infusion, the composition comprising DGEA is generally suspended. This can be done in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate-acetic acid buffers. A form of repository or “depot” slow release preparation may be used so that therapeutically effective amounts of the preparation are delivered into the bloodstream over many hours or days following transdermal injection or delivery.

The composition comprising DGEA is also suitably administered by sustained-release systems. Suitable examples of sustained-release formulations include suitable polymeric materials (such as, for example, semi-permeable polymer matrices in the form of shaped articles, e.g., films, or microcapsules), suitable hydrophobic materials (such as, for example, an emulsion in an acceptable oil) or ion exchange resins, and sparingly soluble derivatives (such as, for example, a sparingly soluble salt). Sustained-release formulations may be administered orally, rectally, parenterally, intracistemally, intravaginally, intraperitoneally, topically (as by powders, ointments, gels, drops or transdermal patch), bucally, or as an oral or nasal spray.

Preparations for administration can be suitably formulated to give controlled release of the composition comprising DGEA over an extended period of time. For example, the pharmaceutical compositions may be in the form of particles comprising a biodegradable polymer and/or a polysaccharide jellifying and/or bioadhesive polymer, an amphiphilic polymer, an agent modifying the interface properties of the particles and a pharmacologically active substance. These compositions exhibit certain biocompatibility features which allow a controlled release of the active substance. See, for example, U.S. Pat. No. 5,700,486.

For oral administration, the composition comprising DGEA can take the form of, for example, the composition comprising DGEA can be included in tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art.

The pharmaceutically acceptable carriers and excipients useful in these methods are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate. Actual methods of preparing such dosage forms are known, or will be apparent, to those skilled in the art.

Generally, the formulations are prepared by contacting the composition comprising DGEA uniformly and intimately with liquid carriers or finely divided solid carriers or both. Then, if necessary, the product is shaped into the desired formulation. Optionally, the carrier is a parenteral carrier, and in some embodiments it is a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The pharmaceutical compositions that comprise the composition comprising DGEA, in some embodiments, will be formulated in unit dosage form, suitable for individual administration of precise dosages. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated. Multiple treatments are envisioned, such as over defined intervals of time, such as daily, bi-weekly, weekly, bi-monthly or monthly, such that chronic administration is achieved. As disclosed herein, therapeutically effective amounts of the composition comprising DGEA are of use for preventing development of an inflammatory reaction such as arthritis, asthma or an allergic reaction, or for treating these disorders. Administration may begin whenever the regression or prevention of disease is desired, for example, at a certain age of a subject, or prior to an environmental exposure.

The composition comprising DGEA can be administered in conjunction with a steroidal anti-inflammatory agent or a non-steroidal anti-inflammatory agent. Steroidal anti-inflammatory agents include glucocorticoids, dexamethasone, prednisone, and hydrocortisone. Non steroidal anti-inflammatory agents include Salicylates (such as Acetylsalicylic acid (Aspirin), Amoxiprin, Benorylate/Benorylate, Choline magnesium salicylate, Diflunisal, Ethenzamide, Fisalamine, Methyl salicylate, Magnesium salicylate, Salicyl salicylate. Salicylamide) Arylalkanoic acids (such as Diclofenac, Aceclofenac, Acemetacin, Alclofenac Bromfenac, Etodolac, Indomethacin, Nabumetone, Oxametacine, Proglumetacin, Sulindac, Tolmetin), 2-Arylpropionic acids (such as Ibuprofen, Alminoprofen, Carprofen, Dexibuprofen, Dexketoprofen, Fenbufen, Fenoprofen, Flunoxaprofen, Flurbiprofen, Ibuproxam, Indoprofen, Ketorolac, Loxoprofen, Naproxen Oxaprozin, Pirprofen, Suprofen, Tiaprofenic acid), N-Acylanthranilic acids (such as Mefenamic acid, Flufenamic acid, Meclofenamic acid, Tolfenamic acid) Pyrazolidine derivatives (such as Phenylbutazone, Ampyrone, Azapropazone, Clofezone, Kebuzone, Metamizole, Mofebutazone, Oxyphenbutazone, Phenazone, Sulfinpyrazone) Oxicams (such as Piroxicam, Droxicam, Lornoxicam, Meloxicam, Tenoxicam or COX-2 inhibitors.

### IV. Inhibiting Macrophage Activation

In certain embodiments of the methods disclosed herein for inhibiting activation of pro-inflammatory M1 macrophages, comprising contacting one or more macrophages with a composition comprising DGEA, the inhibiting activation of pro-inflammatory M1 macrophages comprises suppressing the one or more macrophages from undergoing polarization to a M1 pro-inflammatory phenotype. In certain embodiments, the one or more macrophages to be contacted are unactivated (M0 macrophages).

Macrophages play an important role in both innate and adaptive immunity by activating T lymphocytes. Macrophages that activate Th1 T lymphocytes provide an inflammatory response (pro-inflammatory) and are denoted M1 macrophages. M1 macrophages, also referred to as “killer macrophages,” inhibit cell proliferation, cause tissue damage, and are aggressive against bacteria. Macrophages that activate Th2 T lymphocytes provide an anti-inflammatory response and are denoted M2 macrophages. M2 macrophages, also referred to as “repair macrophages,” promote cell proliferation and tissue repair and are anti-inflammatory. In certain embodiments, as used herein, the terms “non-activated macrophages,” “unactivated macrophages,” “unpolarized macrophages,” or “M0 macrophages” refer to macrophages that have not acquired the phenotype characteristics typical of M1 and M2 macrophage subtypes that are known in the art.

In certain embodiments of the method of inhibiting activation of pro-inflammatory M1 macrophages, the method comprises suppressing expression of inducible nitric oxide synthase (iNOS). In certain embodiments, the method suppresses expression of inducible nitric oxide synthase (iNOS) by at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Methods for measuring the suppression of inducible nitric oxide synthase (iNOS) are described herein, inter alia, in the drawings and examples.

In certain embodiments of the method of inhibiting activation of pro-inflammatory M1 macrophages, the method comprises suppressing expression of Tumor Necrosis Factor alpha (TNFα). In certain embodiments, the method suppresses expression of Tumor Necrosis Factor alpha (TNFα) by at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Methods for measuring the suppression of Tumor Necrosis Factor alpha (TNFα) are described herein, inter alia, in the drawings and examples.

In certain embodiments of the method of inhibiting activation of pro-inflammatory M1 macrophages, the method comprises suppressing expression of Interleukin 6 (IL-6). In certain embodiments, the method suppresses expression of Interleukin 6 (IL-6) by at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Methods for measuring the suppression of Interleukin 6 (IL-6) are described herein, inter alia, in the drawings.

In certain embodiments of the method of inhibiting activation of pro-inflammatory M1 macrophages, the contacting is carried out ex vivo, in vitro, or in vivo.

In certain embodiments of the method of inhibiting activation of pro-inflammatory M1 macrophages, the source of the macrophages is tissue at or near a site of inflammation in a subject or a culture medium comprising monocytes. In certain embodiments, the source of macrophages can be an isolated source, which comprises an ex-vivo composition comprising macrophages. Such a composition may be a culture of macrophages, a macrophage-containing tissue obtained from a subject (which may be the subject to be treated), or a culture, such as a culture comprising monocytes. In certain embodiments of the method of inhibiting activation of pro-inflammatory M1 macrophages, the source of the macrophages is tissue at or near a site of inflammation in a subject or a culture medium comprising monocytes, wherein the macrophages are unactivated (M0) macrophages. As used herein, “one or more macrophages” refers to a population of macrophages, the source of which can be, for example, tissue at or near a site of inflammation in a subject or a culture medium comprising monocytes.

In certain embodiments, the subject matter disclosed herein is directed to a method of inhibiting activation of pro-inflammatory M1 macrophages in a subject in need thereof, comprising administering to the subject a therapeutically effective amount of a composition comprising DGEA.

In certain embodiments of the method of inhibiting activation of pro-inflammatory M1 macrophages in a subject in need thereof, the method is for use in treating an inflammatory disease in a subject in need thereof. In certain embodiments of the method of inhibiting activation of pro-inflammatory M1 macrophages in a subject in need thereof, the subject is suffering from an inflammatory disease.

In certain embodiments of the method of inhibiting activation of pro-inflammatory M1 macrophages in a subject in need thereof, the subject is suffering from conditions associated with undesirable M1 polarization, and wherein the administering suppresses polarization of one or more macrophages to a M1 pro-inflammatory phenotype. In certain embodiments, the one or more macrophages are unactivated (M0) macrophages and they are suppressed from undergoing polarization to a M1 macrophage phenotype. In certain embodiments, a subject that is suffering from conditions associated with undesirable M1 polarization is suffering from chronic or excessive inflammation, which are conditions described herein.

The subject matter described herein is directed to the following embodiments:
1. A crosslinked poly(alkylene glycol)-based hydrogel composition, comprising:
   a cell adhesive peptide covalently conjugated with a first poly(alkylene glycol);
   a cleavable peptide linker covalently conjugated with a second and third poly(alkylene glycol); and
   DGEA (SEQ ID NO: 12) covalently conjugated with a fourth poly(alkylene glycol);
wherein, said first, second, third, and fourth poly(alkylene glycol), in each instance, are the same or different.
2. The crosslinked poly(alkylene glycol)-based hydrogel composition of embodiment 1, wherein said cell adhesive peptide in said cell adhesive peptide covalently conjugated with said first poly(alkylene glycol) is selected from the group consisting of RGDS (SEQ ID NO: 4), RDGS (SEQ ID NO: 5), RGES (SEQ ID NO: 6), REGS (SEQ ID NO: 7), IKVAV (SEQ ID NO: 8), VVIAK (SEQ ID NO: 9), YIGSR (SEQ ID NO: 10), YSRIG (SEQ ID NO: 11), DAEG (SEQ ID NO: 13), and combinations thereof.
3. The crosslinked poly(alkylene glycol)-based hydrogel composition of embodiment 1 or 2, wherein said cell adhesive peptide is RGDS (SEQ ID NO: 4).
4. The crosslinked poly(alkylene glycol)-based hydrogel composition of any one of embodiments 1-3, wherein said cleavable peptide linker in said cleavable peptide linker covalently conjugated with a second and third poly(alkylene glycol) is selected from the group consisting of GGGPQGIWGQGK (SEQ ID NO: 1), GGGIQQWGPGGK (SEQ ID NO: 2), and GGGGGIPQQWGK (SEQ ID NO: 3).
5. The crosslinked poly(alkylene glycol)-based hydrogel composition of any one of embodiments 1-4, wherein said cleavable peptide linker is GGGPQGIWGQGK (SEQ ID NO: 1).
6. The crosslinked poly(alkylene glycol)-based hydrogel composition of any one of embodiments 1-5, wherein said first, second, third, and fourth poly(alkylene glycol), in each instance, is selected from the group consisting of acrylate-PEG-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succnimidyl succinate (PEG-SS), and acrylate-PEG-maleimide (PEG-MAL).
7. The crosslinked poly(alkylene glycol)-based hydrogel composition of any one of embodiments 1-6, wherein said first, second, third, and fourth poly(alkylene glycol) are acrylate-PEG-succinimidyl valerate (PEG-SVA).
8. The crosslinked poly(alkylene glycol)-based hydrogel composition of any one of embodiments 1-7, wherein the conjugate of each of said cell adhesive peptide covalently conjugated with said first poly(alkylene glycol), said cleavable peptide linker covalently conjugated with said second and third poly(alkylene glycol), and said DGEA (SEQ ID NO: 12) covalently conjugated with said fourth poly(alkylene glycol), comprises:
   an acrylate-PEG cell adhesive peptide conjugate;
   an acrylate-PEG cleavable peptide linker conjugate comprising a first and second acrylate-PEG group, wherein said cleavable peptide linker is disposed between said first and second acrylate-PEG group; and
   an acrylate-PEG DGEA conjugate.
9. The crosslinked poly(alkylene glycol)-based hydrogel composition of any one of embodiments 1-8, wherein said cell adhesive peptide covalently conjugated with a first poly(alkylene glycol) is present in said crosslinked poly(alkylene glycol)-based hydrogel composition at a concentration of about 0.5 mM to about 10 mM.
10. The crosslinked poly(alkylene glycol)-based hydrogel composition of any one of embodiments 1-9, wherein said cell adhesive peptide covalently conjugated with a first poly(alkylene glycol) is present in said crosslinked poly(alkylene glycol)-based hydrogel composition at a concentration of about 3.5 mM.
11. The crosslinked poly(alkylene glycol)-based hydrogel composition of any one of embodiments 1-10, wherein said DGEA (SEQ ID NO: 12) covalently conjugated with said fourth poly(alkylene glycol) is present in said crosslinked poly(alkylene glycol)-based hydrogel composition at a concentration of about 1 mM to about 15 mM.
12. The crosslinked poly(alkylene glycol)-based hydrogel composition of any one of embodiments 1-11, wherein said DGEA (SEQ ID NO: 12) covalently conjugated with said fourth poly(alkylene glycol) is present in said crosslinked poly(alkylene glycol)-based hydrogel composition at a concentration of about 5 mM.
13. The crosslinked poly(alkylene glycol)-based hydrogel composition of any one of embodiments 1-12, wherein said cleavable peptide linker covalently conjugated with a second and third poly(alkylene glycol) is present in said crosslinked poly(alkylene glycol)-based hydrogel at about 2 wt % to about 15 wt %.
14. The crosslinked poly(alkylene glycol)-based hydrogel composition of any one of embodiments 1-13, wherein said cleavable peptide linker covalently conjugated with a second and third poly(alkylene glycol) is present in said crosslinked poly(alkylene glycol)-based hydrogel at about 5 wt %.
15. A method of treating a disease or disorder associated with excessive or sustained inflammation in a subject in need thereof, comprising administering to said subject a therapeutically effective amount of a composition comprising DGEA.
16. The method of embodiment 15, wherein said composition comprising DGEA is a hydrogel composition comprising DGEA and one or more synthetic polymers.
17. The method of embodiment 15 or 16, wherein said hydrogel composition comprising DGEA and one or more synthetic polymers is the crosslinked poly(alkylene glycol)-based hydrogel composition of any one of embodiments 1-14.
18. The method of any one of embodiments 15-17, wherein said inflammatory disease or disorder is selected from the group consisting of arthritis, asthma, inflammatory bowel disease, chronic obstructive pulmonary disease (COPD), allergic rhinitis, vasculitis, inflammatory neuropathy, psoriasis, systemic lupus erythematosis (SLE), chronic thyroiditis, Hashimoto's thyroiditis, Addison's disease, polymyalgia rheumatica, Sjögren's syndrome, and Churg-Strauss syndrome.
19. The method of any one of embodiments 15-18, wherein said composition comprising DGEA is administered topically to said subject.
20. A method of inhibiting activation of pro-inflammatory M1 macrophages, comprising contacting one or more macrophages with a composition comprising DGEA.
21. The method of embodiment 20, wherein said inhibiting activation of pro-inflammatory M1 macrophages comprises suppressing said one or more macrophages from undergoing polarization to a M1 pro-inflammatory phenotype.
22. The method of embodiment 20 or 21, wherein said method of inhibiting activation of pro-inflammatory M1 macrophages comprises suppressing expression of inducible nitric oxide synthase (iNOS).
23. The method of embodiment 22, wherein said method suppresses expression of inducible nitric oxide synthase (iNOS) by at least 50%.
24. The method of embodiment 22, wherein said method suppresses expression of inducible nitric oxide synthase (iNOS) by at least 75%.
25. The method of embodiment 20, wherein said method of inhibiting activation of pro-inflammatory M1 macrophages comprises suppressing expression of Tumor Necrosis Factor alpha (TNFα).
26. The method of embodiment 20, wherein said method of inhibiting activation of pro-inflammatory M1 macrophages comprises suppressing expression of Interleukin 6 (IL-6).
27. The method of any one of embodiments 20-26, wherein said contacting is carried out ex vivo, in vitro, or in vivo.
28. The method of any one of embodiments 20-27, wherein said composition comprising DGEA is a hydrogel composition comprising DGEA and one or more synthetic polymers.
29. The method of embodiment 28, wherein said hydrogel composition comprising DGEA and one or more synthetic polymers is the crosslinked poly(alkylene glycol)-based hydrogel composition of any one of embodiments 1-14.
30. The method of any one of embodiments 20-29, wherein the source of said one or more macrophages is tissue at or near a site of inflammation in a subject or a culture medium comprising monocytes.
31. A method of inhibiting activation of pro-inflammatory M1 macrophages in a subject in need thereof, comprising administering to the subject a therapeutically effective amount of a composition comprising DGEA.
32. The method of embodiment 31, wherein said composition comprising DGEA is a hydrogel composition comprising DGEA and one or more synthetic polymers.
33. The method of embodiment 32, wherein said hydrogel composition comprising DGEA and one or more synthetic polymers is the crosslinked poly(alkylene glycol)-based hydrogel composition of any one of embodiments 1-14.
34. The method of embodiment 31, wherein the subject is suffering from an inflammatory disease.
35. The method of embodiment 31, wherein the subject is suffering from conditions associated with undesirable M1 polarization, and wherein said administering suppresses polarization of one or more macrophages to a M1 pro-inflammatory phenotype.

The following examples are offered by way of illustration and not by way of limitation.

### Examples

Non-resolving, persistent inflammation contributes to wound chronicity, leading to a myriad of ailments. Macrophage polarization can guide the transition of the immune response away from inflammation towards regeneration (Y. Liu, T. Segura, “Biomaterials-mediated regulation of macrophage cell fate,” Front. Bioeng. Biotechnol. 8, 1 (2020)). Macrophage polarization can be engineered by designing immunomodulating biomaterials. A unique biomaterial design was developed, as shown herein, using a collagen-derived peptide, DGEA, which can inhibit M1 macrophage polarization. As demonstrated in the Examples below, DGEA can be used as a soluble factor in 2D cultures, as well as covalently bound with PEG in 3D hydrogels, to successfully interfere with M1 macrophage phenotype.

### Example 1: Soluble Delivery of DGEA to 2D Cultures of Macrophages

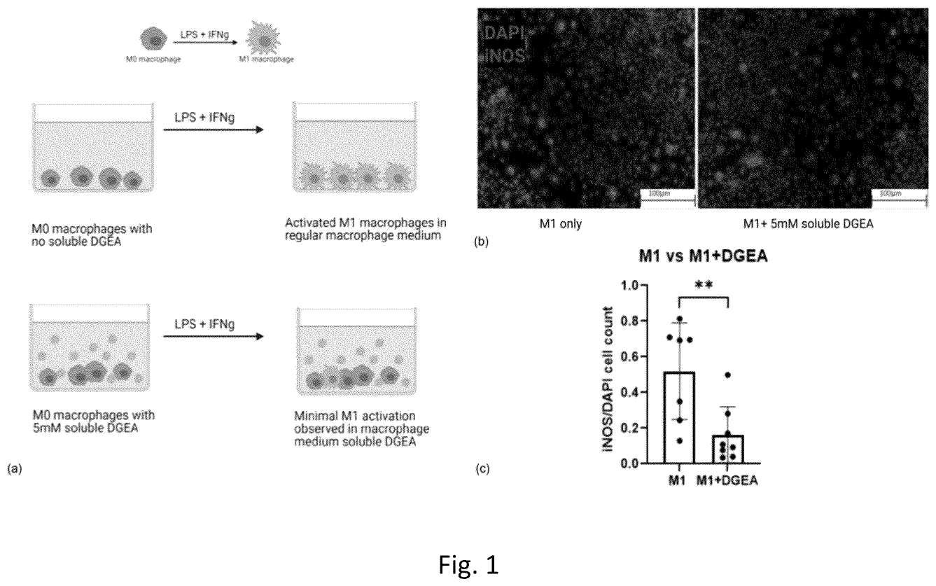

To establish interactions between DGEA and M1 macrophages, a 2D study was conducted with soluble delivery of DGEA to the media culture. Macrophages were stimulated to the M1 phenotype by addition of LPS and IFNγ, as can be seen in inset (a) of FIG. 1. 5 mM DGEA was dissolved in the media to assess the effects of DGEA on macrophage activation (S. Y. Yoo, M. Kobayashi, P. P. Lee, and S. W. Lee, “Early osteogenic differentiation of mouse preosteoblasts induced by collagen-derived DGEA-peptide on nanofibrous phage tissue matrices,” Biomacromolecules, vol. 12, no. 4, pp. 987-996, April 2011). After stimulation, the cells were stained for 4′,6-diamidino-2-phenylindole (DAPI) and iNOS (inset (b) of FIG. 1). The addition of 5 mM DGEA in the media reduced the number of iNOS⁺ cells, as represented in insets (b) and (c) in FIG. 1. The iNOS⁺ cell count was normalized to the number of DAPI⁺ cells for each image chosen to be analyzed. Performing a Student's t-test on the data demonstrated that the conditions were statistically different (p<0.05). Polarized iNOS⁺ macrophage cells with soluble DGEA were dramatically lower than iNOS⁺ macrophage cells without any presence of DGEA. Quantifying the graphs in inset (c) in FIG. 1 shows that iNOS⁺ activated macrophages were 0.5±0.1 fraction of the total DAPI⁺ cells whereas, M1 activated macrophages in the presence of DGEA were 0.1±0.1 fraction of the total DAPI+ cells. These results verify that DGEA can act as an inhibitor in polarizing M1 macrophages. ECM-derived peptides have been investigated in cell function. For example, the interaction between collagen and α2β1 integrin is important for the osteoblastic differentiation of bone marrow cells. As shown by Mizuno et al., the addition of DGEA peptide to a culture of bone marrow cells encapsulated in type I collagen matrix gels inhibited the expression of osteoblastic phenotype of bone marrow cells (M. Mizuno, R. Fujisawa, and Y. Kuboki, “Type I collagen-induced osteoblastic differentiation of bone-marrow cells mediated by collagen-α2β1 integrin interaction,” J Cell. Physiol., 2000). Previous work has also suggested DGEA to be a potential blocker for α2β1, particularly Luzak et al. demonstrated significant inhibition in platelet adhesion, as DGEA blocked surface receptors interacting with collagen (B. Luzak, J. Golanski, M. Rozalski, M. A. Boncler, and C. Watala, “Inhibition of collagen-induced platelet reactivity by DGEA peptide Circled white star,” Acta Biochim. Pol., 2003). Fishman et al. demonstrated DGEA to block adhesion of ovarian carcinoma cells to collagen (S. Y. Yoo, M. Kobayashi, P. P. Lee, and S. W. Lee, “Early osteogenic differentiation of mouse preosteoblasts induced by collagen-derived DGEA-peptide on nanofibrous phage tissue matrices,” Biomacromolecules, vol. 12, no. 4, pp. 987-996, April 2011). Furthermore, Cha et al. showed an upregulation of α2β1 by M1 macrophages (B. H. Cha et al., “Integrin-Mediated Interactions Control Macrophage Polarization in 3D Hydrogels,” Adv. Healthc. Mater., 2017). Thus, the reduction in iNOS+ cells upon culture with DGEA is within the context of established literature.

### Example 2: Soluble Delivery of DGEA in a 3D Matrix of PEG Hydrogels

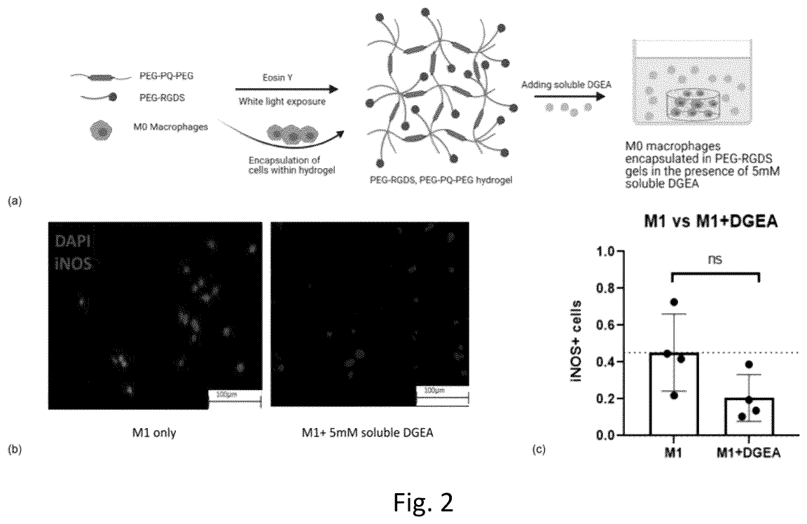

The tunable nature of polyethylene glycol (PEG), a synthetic polymer, makes it a valuable material scaffold in tissue engineering (E. B. Peters, N. Christoforou, K. W. Leong, G. A. Truskey, and J. L. West, “Poly(Ethylene Glycol) Hydrogel Scaffolds Containing Cell-Adhesive and Protease-Sensitive Peptides Support Microvessel Formation by Endothelial Progenitor Cells,” Cell. Mol. Bioeng., vol. 9). An acrylate-PEG-succinimidyl valerate (acryl-PEG-SVA) was used. To immobilize the peptide, the peptide was reacted with acryl-PEG-SVA. Via amine substitution, the product is acrylate-PEG-peptide. The acrylate group on the end of the acrylate-PEG-peptide chain allows for immobilization into the crosslinked hydrogel. While PEG is hydrophilic, these acrylate groups are hydrophobic in nature, creating micelle-like centers in which free radicals rapidly propagate after initiation (D. L. Hem, J. A. Hubbell, “Incorporation of adhesion peptides into nonadhesive hydrogels useful for tissue resurfacing,” J Biomed. Mater. Res. 39, 266-276 (1998); G. Zhou, F. Khan, Q. Dai, J. E. Sylvester, and S. J. Kron, “Photocleavable peptide-oligonucleotide conjugates for protein kinase assays by MALDI-TOF MS,” Mol. Biosyst., 2012). To further evaluate whether soluble delivery of DGEA has the same inhibitory effect on M1 macrophage polarization in a 3D microenvironment, an ECM-mimicking PEG-based hydrogel was developed (E. M. Moore, “Investigating the roles of macrophages in vessel development utilizing poly(ethylene glycol) hydrogels.” (2018); L. M. Weber, K. N. Hayda, K. Haskins, and K. S. Anseth, “The effects of cell-matrix interactions on encapsulated β-cell function within hydrogels functionalized with matrix-derived adhesive peptides,” Biomaterials, 2007; Y. Wu, K. Jane Grande-Allen, and J. L. West, “Adhesive Peptide Sequences Regulate Valve Interstitial Cell Adhesion, Phenotype and Extracellular Matrix Deposition,” Cell. Mol. Bioeng., 2016; G. Zhou, F. Khan, Q. Dai, J. E. Sylvester, and S. J. Kron, “Photocleavable peptide-oligonucleotide conjugates for protein kinase assays by MALDI-TOF MS,” Mol. Biosyst., 2012). With PEG as its backbone, this hydrogel contained a cell-adhesive component RGDS (Arg-Gly-Asp-Ser) (SEQ ID NO: 4) and an enzyme-cleavable component GGGPQGIWGQGK (SEQ ID NO: 1), abbreviated as PQ. This is referred to as the control hydrogel (inset (a) in FIG. 2). While fibronectin derived RGDS provides sites for cell adhesion, PQ is a matrix metalloprotease (MMP-2/9)-sensitive peptide from the collagen chain that is cleaved in the presence of MMPs −2 and −9. This enzyme-specific cleavage allows for cell-mediated migration through the PEG-based hydrogel (J. J. Moon et al., “Biomimetic hydrogels with pro-angiogenic properties,” Biomaterials 31(14), 3840-3847 (2010)). The incorporation of this enzyme-cleavable component mimics the natural ECM (J. L. West, J. A. Hubbell, “Polymeric biomaterials with degradation sites for proteases involved in cell migration,” Macromolecules 32(1), 241-244 (1999)). The PEG macromers, acrylate-PEGRGDS, and acrylate-PEG-PQ-PEG-acrylate are mixed with cells, photoinitiator eosin Y, and N-Vinylpyrrolidone (NVP). Following white light exposure, free radicals are generated and propagate in the micelle-like acrylate centers, thus, allowing crosslinking and rapid polymerization of the hydrogel.

Raw 264.7 macrophages were encapsulated within the hydrogel. These quiescent M0 macrophages were allowed to equilibrate in the incubator for 24 hrs, after which M1 media was added to stimulate them towards the pro-inflammatory phenotype. At this time, 5 mM DGEA was also dissolved into the media and the samples were incubated for another 72 hrs. The samples were then fixed, stained, imaged, and analyzed. The immunofluorescent images represent DAPI stained cells and iNOS stained cells (inset (b) in FIG. 2). The 3D hydrogels were imaged using a Keyence BZ-X800 microscope and the images represent 2D slices of an entire 3D Z-stack. The Student's t-test shown in inset (c) in FIG. 2 revealed similar trends when compared with inset (c) in FIG. 1, i.e. the number of iNOS⁺ cells in the presence of soluble DGEA was lower than the number of iNOS⁺ cells without any DGEA. The data were statistically non-significant (p>0.05). This non-significance may be due to improper diffusion of the DGEA peptide into the hydrogel, thus, minimizing exposure of the encapsulated cells to DGEA treatment. However, similar trends in reduction of M1 macrophage were observed, suggesting that DGEA can reduce M1 polarization via soluble delivery in a PEG hydrogel.

### Example 3: Immobilizing DGEA in a PEG Hydrogel and Assessing Conjugation

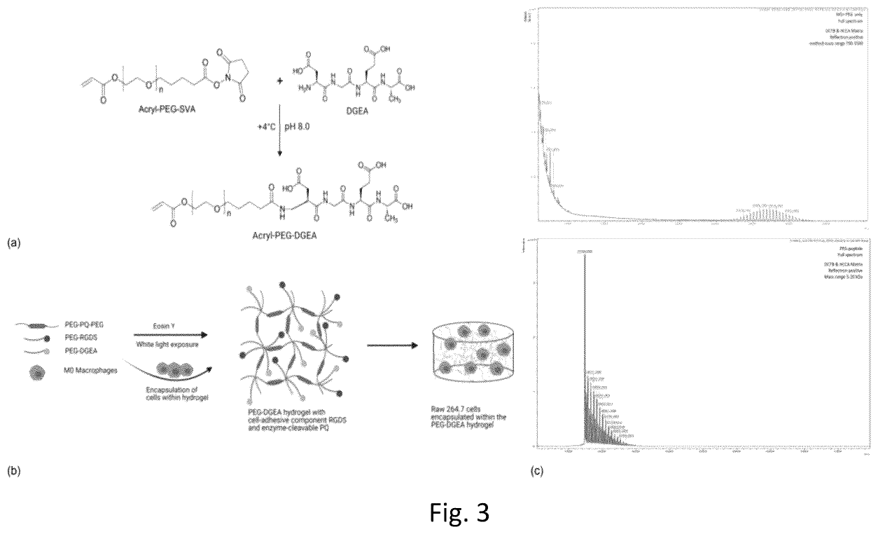

A biomaterial with ECM-mimicking characteristics was designed by immobilizing DGEA in the hydrogel environment. The control hydrogel was an ECM-mimicking PEG hydrogel conjugated with RGDS (SEQ ID NO: 4), the cell-adhesive component, and PQ, the enzyme-cleavable component. A biomaterial was designed that incorporated DGEA in the control hydrogel, in the form of PEG-DGEA. The chemical formulations of the organic compounds are as depicted in inset (a) in FIG. 3. DGEA and other ECM-derived peptides such as laminin-derived IKVAV and YIGSR have been used covalently with PEG in the form of a biofunctionalized hydrogel for purposes such as encapsulating islets to promote cell viability or investigating the effects of the peptides on valve interstitial cells (L. M. Weber, K. N. Hayda, K. Haskins, K. S. Anseth, “The effects of cell-matrix interactions on encapsulated β-cell function within hydrogels functionalized with matrix-derived adhesive peptides,” Biomaterials 28, 3004-3011 (2007); Y. Wu, K. Jane Grande-Allen, J. L. West, “Adhesive peptide sequences regulate valve interstitial cell adhesion, phenotype and extracellular matrix deposition,” Cell. Mol. Bioeng. 9, 479-495 (2016)).

MALDI-ToF was applied to monitor the conjugation of PEG with DGEA (inset (c) in FIG. 3) (G. Zhou, F. Khan, Q. Dai, J. E. Sylvester, S. J. Kron, “Photocleavable peptide-oligonucleotide conjugates for protein kinase assays by MALDI-TOF MS,” Mol. Biosyst. 8, 2404 (2012); J. Kemptner, M. Marchetti Deschmann, J. Siekmann, P. L. Turecek, H. P. Schwarz, G. Allmaier, “GEMMA and MALDI-TOF MS of reactive PEGs for pharmaceutical applications,” J Pharm. Biomed. Anal. 52, 432-437 (2010). The x-axis represents the mass-to-charge ratio (m/z), while the y-axis represents intensity in fluorescence arbitrary units. The molecular weight of the PEG monomer (Acryl-PEG-SVA) is 3400 g/mol, inset (c) in FIG. 3, top right, as is displayed by a dominant peak closer to the origin of the x and y axes. The molecular weight of DGEA is 390.35 g/mol. The shift in peak from PEG to PEG-DGEA inset (c) in FIG. 3, bottom right, is evident as the higher molecular weight of PEG-DGEA (MW=3790.5 g/mol), pushed the peak further away from the origin of both axes. These results indicate successful conjugation of PEG-DGEA. Cells were encapsulated in the PEG-DGEA hydrogel or control hydrogel as described below.

### Example 4: Immobilized DGEA in a PEG Hydrogel Platform has Inhibitory Effects on M1 Macrophage Polarization

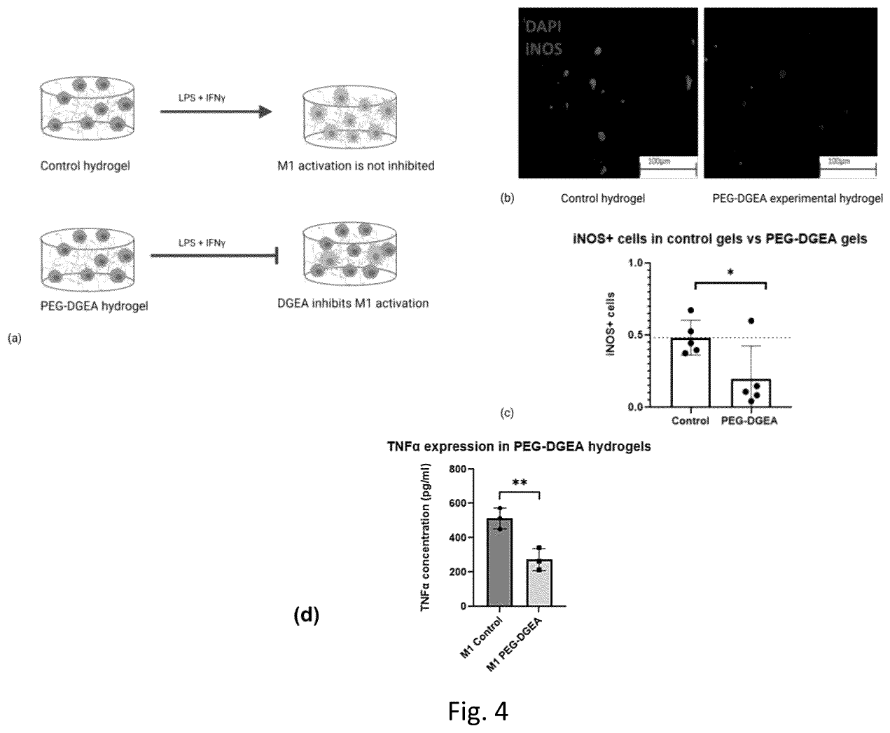

The M1 response was next assessed in a 3D environment with immobilized DGEA. A study by Mehta et al. highlighted the role of DGEA immobilized in alginate hydrogels to induce an osteogenic phenotype in mesenchymal stem cells (M. Mehta, C. M. Madl, S. Lee, G. N. Duda, and D. J. Mooney, “The collagen i mimetic peptide DGEA enhances an osteogenic phenotype in mesenchymal stem cells when presented from cell-encapsulating hydrogels,” J Biomed. Mater. Res.—Part A, 2015). Employing the PEG-DGEA hydrogel, as described above, and control hydrogel as the experimental and control groups respectively, M0 macrophages were encapsulated in each gel (inset (a) in FIG. 4). M1 media was added 24 hrs post-encapsulation to stimulate M0 macrophages towards the M1 phenotype. 72 hrs following the addition of the M1 media, cells were fixed, stained, and analyzed. The immunostaining of cells using iNOS and DAPI is represented in inset (b) of FIG. 4. M1 macrophages encapsulated in the control hydrogels exhibited 0.5±0.1 iNOS⁺ cells per total DAPI⁺ cells. Conversely, M1 macrophages in PEG-DGEA hydrogels had a ratio of less than 0.2±0.1 of iNOS⁺ cells/DAPI⁺ cells. Student's t-tests were performed to evaluate if the data were statistically significant (inset (c) in FIG. 4. The results of the statistical analysis reflected the visual observations from the images of stained samples. The data are statistically different for both conditions. Further, conditioned media from the encapsulated M1 macrophages was collected for ELISA analysis of soluble cytokine secretion. TNFα is a pro-inflammatory cytokine robustly secreted by M1 macrophages (R. Lv, Q. Bao, Y. Li, “Regulation of M1-type and M2-type macrophage polarization in RAW264.7 cells by galectin-9,” Mol. Med. Rep. 16(6), 9111-9119 (2017)). Inset (d) in FIG. 4 demonstrates a significant reduction in the expression of TNFα by M1 macrophages encapsulated in PEG-DGEA hydrogels for 72 h. This reduction contributes to the idea that in the 3D in vitro environment of PEG-DGEA hydrogels, M1 macrophages can be less inflammatory. The studies demonstrate that the PEG-DGEA hydrogel has inhibitory effects on M1 macrophage polarization.

### Example 5: PEG-DGEA has Inhibitory Effects on Activation of Human-Derived M1 Macrophages

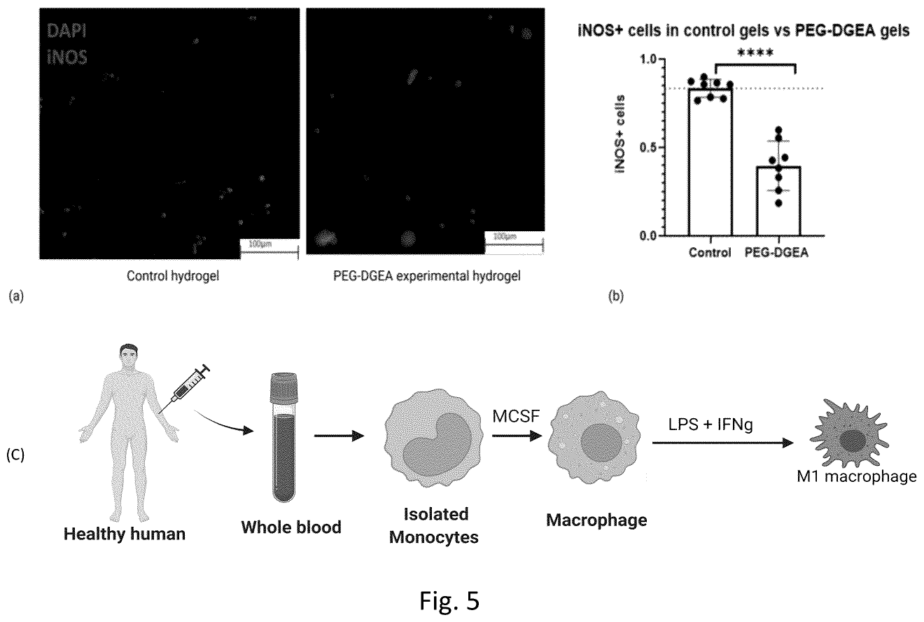

To assess if PEG-DGEA's inhibitory effects on iNOS expression in murine macrophages could be translated to human macrophages, monocytes were isolated from a healthy human blood sample. The monocytes were converted to macrophages on addition of RPMI-1640 supplementary media. These macrophages were encapsulated in control and PEG-DGEA hydrogels as described above. Following encapsulation, human macrophages were stimulated from M0 to the M1 phenotype by adding LPS and IFNγ in the RPMI medium. Samples were stained for iNOS and DAPI for all conditions (inset (a) in FIG. 5). A Student's t-test revealed a significant difference between iNOS⁺ cells for control hydrogels versus iNOS⁺ cells for experimental hydrogels. In particular, 0.85±0.1 iNOS⁺ cells per total DAPI+ cells were observed in the control conditions, and 0.4±0.1 iNOS⁺ cells per total DAPI⁺ cells were observed in the PEG-DGEA hydrogels. These results suggest effective human translation of the PEG-DGEA hydrogel to control inflammatory conditions by manipulating the M1 macrophage phenotype.

### Comparison Between Models

To further investigate the hypothesis of DGEA influencing macrophage polarization, soluble DGEA was delivered in a 2D culture of Raw 264.7 macrophages. This study demonstrated that DGEA inhibits iNOS expression in M1 macrophages, thus, reducing M1 polarization. Reduction of iNOS following 2D soluble delivery prompted assessment of soluble effects of DGEA in 3D. Thus, a soluble delivery study was conducted with a 3D control PEG hydrogel. 5 mM DGEA peptide was dissolved in the media to assess macrophage response via iNOS expression. As previously explained, the control hydrogel contained RGDS and PQ. The soluble delivery of DGEA in 3D did not statistically inhibit iNOS expression in M1 macrophages. However, the trend displays that soluble delivery of DGEA in 3D did reduce iNOS expression (FIG. 2(c)). It is hypothesized that diffusion of the soluble-delivered DGEA peptide through the crosslinked hydrogel was insufficient to significantly alter function of encapsulated macrophages. Furthermore, even though PEG hydrogels are known to allow for diffusion of nutrients, the literature suggests that the inert state of PEG hydrogels hinders therapeutic efficacy of locally delivered soluble factors targeted to the encapsulated cells (C.-C. Lin, K. S. Anseth, “PEG hydrogels for the controlled release of biomolecules in regenerative medicine,” Pharm. Res. 26(3), 631-643 (2008)). Previous studies have highlighted the importance of immobilizing peptides or therapeutics in PEG hydrogels for optimum drug availability and enhanced interactions with the encapsulated cells. To design a hydrogel in which DGEA is immobilized and covalently crosslinked, PEG was conjugated to DGEA for grafting into the PEG hydrogel. This hydrogel constituted of PEG-DGEA, PEG-RGDS, and PEGPQ-PEG. This hydrogel exposed M1 macrophages to the immobilized DGEA peptide and reduced iNOS expression.

### SUMMARY

Utilizing ECM-derived peptides to design immune-informed biomaterials can inform clinical translation for therapeutics in regenerative medicine. The tunable properties of such biomaterials allow researchers to manipulate cell functions such as preventing inflammation, controlling fibrosis, and promoting tissue healing. The subject matter described herein highlights the development of a biomaterial to inhibit pro-inflammatory macrophage polarization. In particular, what is provided is a proof of concept that DGEA plays a role in inhibiting M1 macrophage polarization.

### Materials and Methods

### Cell Culture and Maintenance

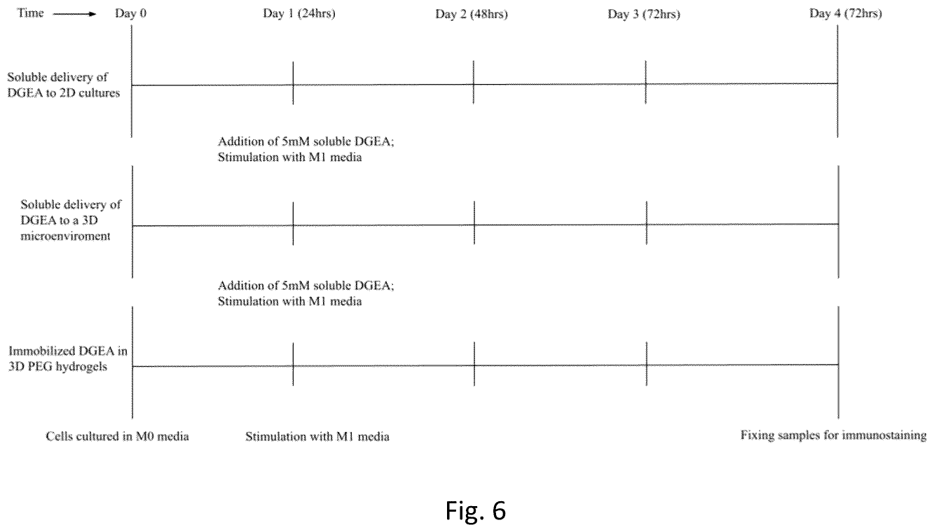

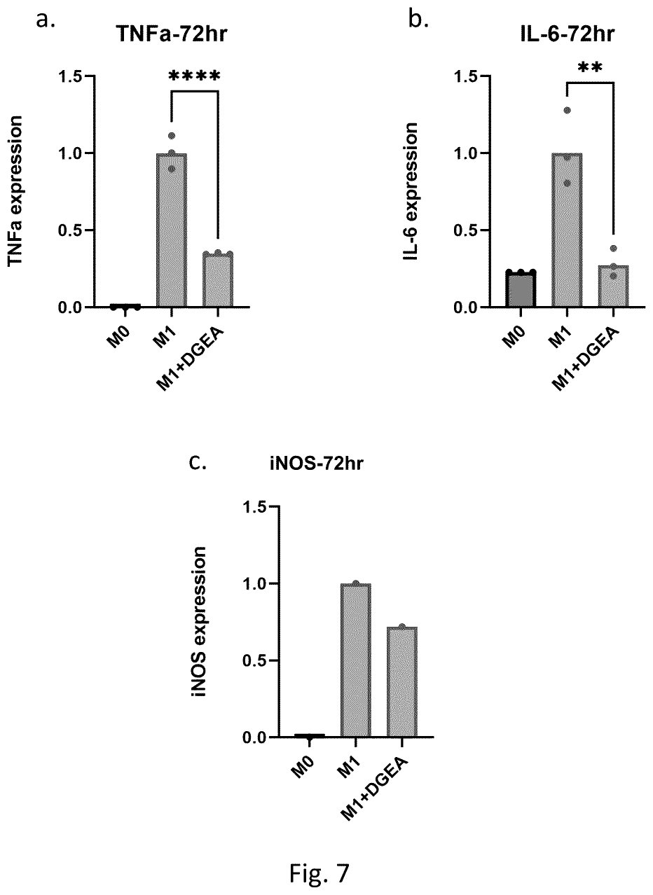

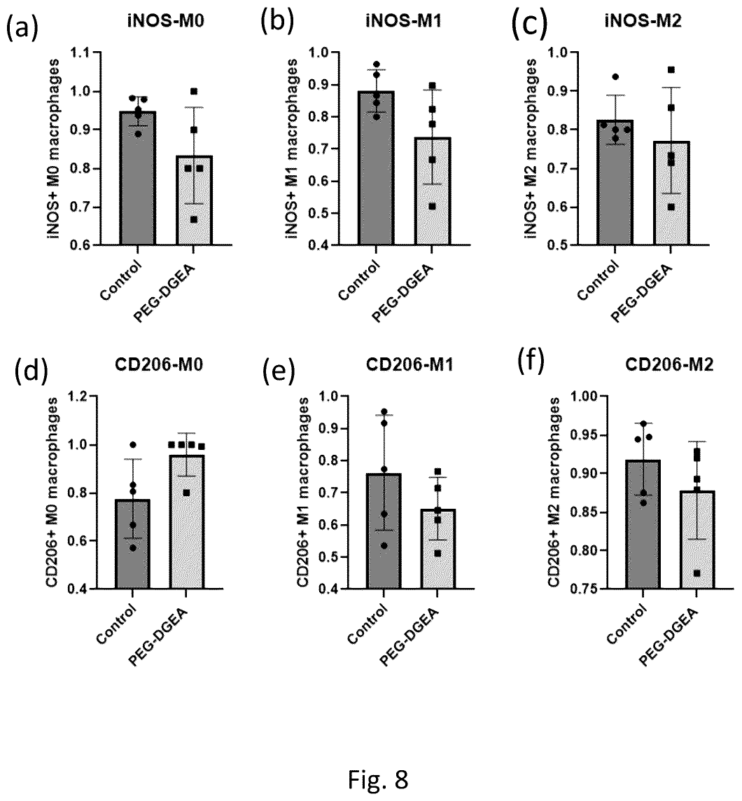

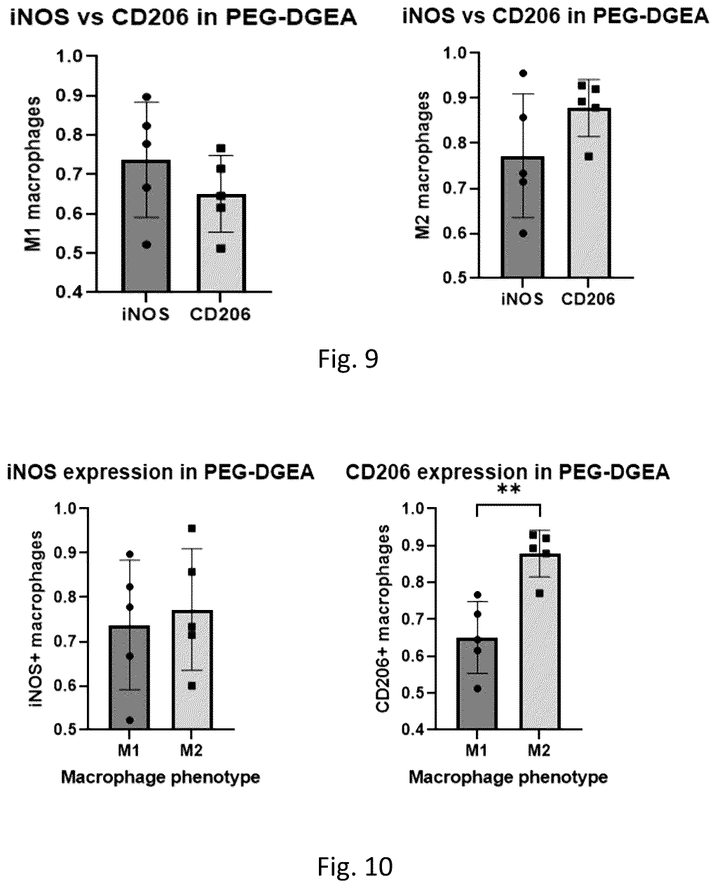

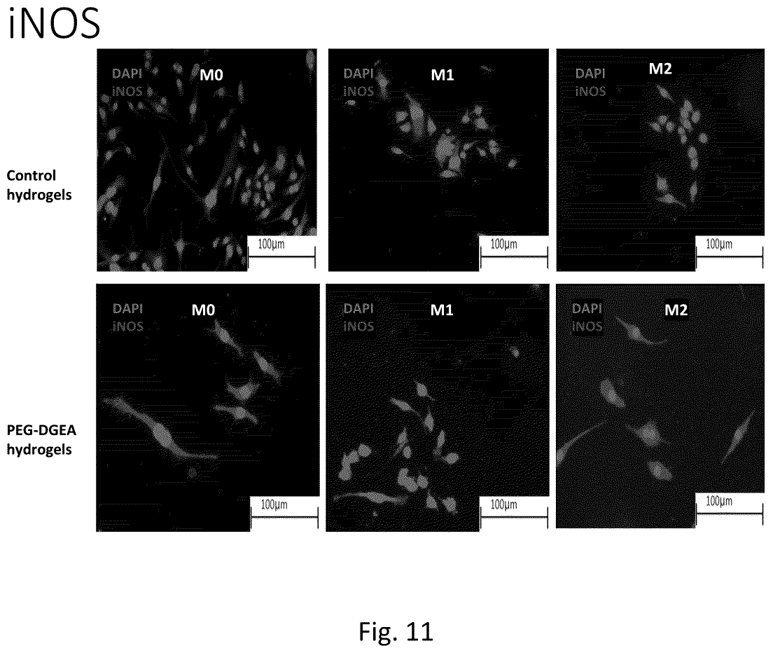

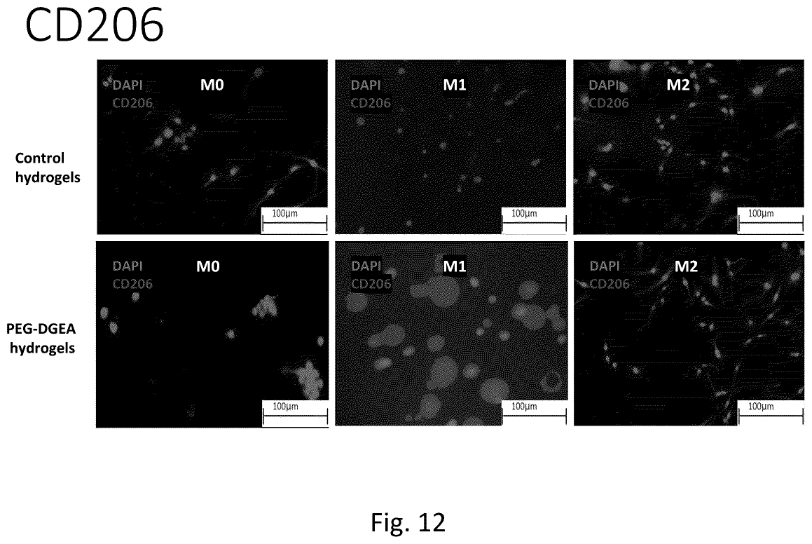

The cell line Raw 264.7, derived from BALB/c mice, was obtained from ATCC. The cells were cultured in Dulbecco's Modified Eagle's Medium (DMEM) (Corning, Corning, NY) supplemented with 10% fetal bovine serum (FBS) (Atlanta Biologicals, Lawrenceville, GA), 100 IU penicillin and 100 μg/mL streptomycin (Corning). This is referred to as M0 media herein. To stimulate the cells towards the M1 phenotype, 10 ng/ml of IFNγ (Prospec, East Brunswick, NJ) along with 100 ng/ml of LPS (Santa Cruz Biotechnology, Dallas, TX) were added to M0 media. This is referred to herein as M1 media. Cells were stimulated to the M1 phenotype 24 hrs post-seeding on a 24-well tissue culture polystyrene (TCP). M0 macrophages were also cultured over the same time periods, resulting in two groups through 72 hours (M0 and M1) (refer to FIG. 6 for experimental design). All cells were maintained at 37° C. in 5% CO₂.

Peripheral blood was obtained in ethylenediaminetetraacetic (EDTA) vacutainer collection tubes from a healthy Caucasian female donor (#IRB202001085) and peripheral blood mononuclear cells (PBMCs) were isolated from whole blood by Ficoll gradient centrifugation. 35 mL blood was diluted 1:1 in calcium/magnesium-free phosphate buffered saline (PBS), slowly layered over 15 mL Ficoll-Paque (GE Healthcare, Piscataway, NJ), and then centrifuged at 400 g for 30 minutes at room temperature with the brakes off. PBMCs were collected and washed twice in PBS by centrifuging at 300 g for 10 minutes. PMBCs were then resuspended in a cell suspension buffer consisting of PBS pH 7.2, 0.5% bovine serum albumin (Fisher Scientific), 2 mM EDTA calcium disodium salt hydrate (TCI America). Monocytes were isolated from PBMCs via magnetic activated cell sorting using a Pan-Monocyte Isolation kit (Milentyi Biotec). Isolated monocytes were collected and washed in 1 ml RPMI-1640 (Gibco, Grand Island, NY). Monocytes were plated on a 6-well tissue culture dish at a density of 5.88×10⁶ cells/mL. To differentiate macrophages from monocytes, RPMI-1640 was supplemented with 2 mM L-glutamine (Gibco), 100 U/ml penicillin (Corning), 100 μg/ml streptomycin (Corning), 0.1 mM sodium pyruvate (Gibco), 1% non-essential amino acids (Gibco), 50 μM 2-mercaptoethanol (Gibco), 10% fetal bovine serum (Atlanta Biologicals), and 20 ng/mL macrophage-colony stimulating factor (M-CSF) (Life Technologies, Carlsbad, CA). Media with M-CSF was changed every 48 hrs post-seeding. All cells were maintained at 37° C. in 5% CO₂. After 5 days of incubation, cells in the M0 state were encapsulated in control and experimental hydrogels, as described above. M1 media was added to the well plates to stimulate macrophages towards the M1 phenotype. This M1 media contained RPMI-1640 with all supplements previously listed, except M-CSF. Instead, 10 ng/ml IFNγ was added along with 100 ng/ml of LPS. Cells were allowed to incubate for 72 hrs for subsequent experiments.

### PEG Hydrogel Fabrication

DGEA (Asp-Gly-Glu-Ala) peptide (obtained from Genscript) was conjugated to PEG by amine substitution reaction of the ECM-derived peptide with acrylate-(poly (ethylene glycol) (PEG)-succinimidyl valerate (SVA) (acrylate-PEG-SVA; Laysan Bio Inc., Arab, AL). A 1.2:1 molar ratio of DGEA peptide to acrylate-PEG-SVA was mixed in 20 mM (N-(2-hydroxyethyl)piperazine-N′-(4-butanesulfonic acid)) (HEPBS) buffer with 100 mM NaCl, 2 mM CaCl₂) and 2 mM MgCl₂ at pH 8.5 (referred to as protein conjugation buffer) (E. M. Moore, G. Ying, and J. L. West, “Macrophages Influence Vessel Formation in 3D Bioactive Hydrogels,” Adv. Biosyst., 2017). The pH of this mixture was then titrated to 8.0 and reacted overnight (16 hr) at 4° C. under constant agitation. The final product (acrylate-PEG-DGEA) was then dialyzed (3.5 kDa molecular weight cut-off MWCO regenerated cellulose; Spectrum Laboratories), lyophilized, and stored at −80° C. (Labconco, Kansas City, MO) until use. The same steps were repeated for cross-linking the cell adhesive component RGDS (Arg-Gly-Asp-Ser) (SEQ ID NO: 4), with PEG to form PEG-RGDS with the molar ratio 1.2:1. A 1:2 molar ratio of PQ (GGGPQGIWGQGK) (SEQ ID NO: 1) peptide to acrylate-PEG-SVA was used for the synthesis of the diacrylate polymer PEG-PQ-PEG. The only difference in the PEG-PQ-PEG conjugation is the molar ratio as well as the dialysis cut-off, the final product was dialyzed at 6 k-8 k MWCO.

The molecular weights of each peptide used are displayed in Table 1:

TABLE 1
Summarized molecular weights and molar ratios of each
peptide used before and after conjugation with PEG.

| Polymer | Molecular | Molar ratio | |
|---|---|---|---|
| modification | weight | (PEG- | Conjugated |
| with peptides | (g/mol) | peptide) | product |
| Acryl-PEG-SVA | 3400 | — | — |
| RGDS | 433.42 | 1:1.2 | PEG-RGDS |
| PQ | 1141 | 2:1 | PEG-PQ-PEG |
| DGEA | 390.35 | 1:1.2 | PEG-DGEA |

Successful conjugation of the DGEA peptide with PEG was confirmed via matrix-assisted laser desorption/ionization-time-of-flight mass spectrometry (MALDI-ToF) (funded by NIH S10 OD021758-01A1). MALDI was performed on Acryl-PEG-SVA, PEG-RGDS, PEG-PQ-PEG, and PEG-DGEA. MALDI matrix DCTB only, HCCA only, and HCCA & DCTB were prepared in a 1:1 ratio of acetonitrile:water with 0.1% Trifluoroacetic acid (TFA). Sample solution was mixed with 3 different MALDI matrices in 1:1 (v/v) ratio. The sample-matrix mixture was spotted on the MALDI plate (1p L) for analysis.

Encapsulation of Cells within PEG Hydrogels

After conjugation, the hydrogels were split into control groups and experimental groups wherein the control group was a PEG-RGDS and PEG-PQ-PEG hydrogel. The experimental condition was a hydrogel construct of PEG-RGDS, PEG-PQ-PEG as well as PEG-DGEA. To form hydrogels, the polymers (2.5% PEG-PQ-PEG and 3.5 mM PEG-RGDS) were dissolved in a HEPES-buffered saline (HBS; 10 mM HEPES and 100 mM NaCl at pH 7.4) with 1.5% triethanolamine (TEOA; Sigma), 10 μM eosin Y and 0.35% (v/v) N-vinyl-pyrrolidone (NVP; Sigma) at pH 8.3. Raw 264.7 macrophages were encapsulated in the hydrogels at 50,000 cells per gel. A 5 μL droplet of the cell-polymer suspension was placed on top of a 385 um PDMS slab, with two PDMS spacers to allow formation of a spheroid 3D gel (E. M. Moore, G. Ying, and J. L. West, “Macrophages Influence Vessel Formation in 3D Bioactive Hydrogels,” Adv. Biosyst., 2017). A methacrylate-modified glass coverslip, bearing groups that permit covalent bonding with the hydrogel, was placed on top of the 5 μL droplet. This was done for ease of handling of the delicate hydrogel and ease of culturing in 24-well plates. The cell-polymer suspension in between the PDMS spacers and the coverslip was exposed to UV light for 60 seconds to allow the droplet to solidify into a hydrogel construct. The coverslip, with the hydrogel facing up, was planted in each well to which media was added in order to supply nutrients to the cells encapsulated within.

### Soluble Delivery of DGEA to Encapsulated Cells in PEG Hydrogels

For soluble delivery of the DGEA peptide in a 3D matrix, 3.5 mM PEG-RGDS and 5% PEG-PQ-PEG hydrogels were made as the control hydrogel. 1 ml M0 media was added to each well of the 24-well plate containing the hydrogels and incubated at 37° C. in 5% CO₂. 24 hrs post encapsulation, M0 media was aspirated and the cells were rinsed with PBS. Wells were split into control and experimental groups. M0 media was added to the control wells and M1 stimulating media was added to the experimental wells (refer to FIG. 6). Media changes occurred at 24 hrs post-encapsulation and subsequently every 48 hrs afterwards. 5 mM DGEA was dissolved into both M0 and M1 media at 0 mM and 5 mM to assess the impact of soluble DGEA on cells in a 3D matrix.

Encapsulating Cells in PEG Hydrogels with Immobilized DGEA

Similarly, for assessing M1 macrophage response to immobilized DGEA, Raw 264.7 cells were encapsulated in a 3D matrix of 5 mM PEG-DGEA, 3.5 mM PEG-RGDS and 5% PEG-PQ-PEG. This has been defined as the experimental hydrogel. 1 ml M0 media was added to each well of the 24-well plate containing the hydrogels. All gels were cultured at 37° C. in 5% CO₂. 24 hrs post encapsulation, M0 media was aspirated and the cells were rinsed with PBS. M1 media was added to all experimental conditions, and control groups were continually cultured in M0 media. Media changes occurred at 24 hrs post-encapsulation and subsequently every 48 hrs afterwards (FIG. 6). The same steps were followed to encapsulate human macrophages in control and experimental hydrogels at a density of 6 million cells/mL (30,000 cells per gel).

### Immunostaining

Immunostaining assays were carried out to analyze the expression levels of iNOS (M1 surrogate marker) and DAPI (nuclei marker) on Raw 264.7 macrophages and human macrophages. Raw 264.7 cells were seeded at 10 million cells/mL onto 24-well plates (50,000 cells per gel). Human macrophages were seeded within the control and experimental hydrogels at 6 million cells/mL (30,000 cells per gel). After being cultured for 72 hours post addition of M1 media, the cells were fixed with 4% paraformaldehyde for 45 mins at room temperature and then washed 3 times with tris buffered saline (TBS). Gels were then permeabilized in 0.25% Triton-X for 45 minutes, rinsed with TBS 4 times, followed by blocking overnight in 5% donkey serum at 4° C. Rinses after blocking took place 3 times in TBS for 5 mins each.

Following blocking, gels were incubated in the primary antibody iNOS (M1 marker) (Rabbit Anti-Mouse Polyclonal Antibody, Invitrogen) at 1:200 in 0.5% DS at 4° C. overnight. Following primary incubation, gels were rinsed 5 times with TBS+0.01% TWEEN for 90-120 minutes. The 5^{th} rinse was left overnight at 4° C. On the consecutive morning, the 6^{th} and final rinse was in TBS alone without the presence of TWEEN. Gels were then incubated overnight at 4° C. with secondary antibody AlexaFluor 555 (Donkey Anti-Rabbit, Life Technologies) at 1:100, following an hour-long rinse with TBS the next day. Cell nuclei were stained with 2 μM 4′,6-diamidino-2-phenylindole (DAPI; nuclear marker). Samples were rinsed twice with TBS for 5 minutes each before imaging.

### Imaging and Image Analysis

Cells were stimulated with M1 media 24 hrs post-seeding and post-encapsulation. In all studies, the effects of DGEA on the cells were assessed 72 hrs post-stimulation with M1 media by immunocytochemistry (ICC). Cells in the soluble studies and encapsulated in gels were imaged using the Keyence BZ-X800 microscope, 72 hours post-stimulation to the M1 phenotype.

Images were quantified based on the number of iNOS⁺ cells, normalized to DAPI⁺ cells for each condition. Images were analyzed using the “automated cell counting of single color image” feature on ImageJ (NIH) software after a randomized, unbiased selection of images. All images were turned to 8-bit grayscale and threshold to highlight all the cells to be counted. Total cell count for each image, recorded as iNOS⁺ and DAPI⁺ cells, were saved in Microsoft Excel and then exported to GraphPad Prism for further statistical analysis.

Enzyme Linked Immunosorbent Assay (ELISA) for TNFα Expression Raw 264.7 cells were encapsulated in control and PEG-DGEA hydrogels to assess differences in TNFα expression due to immobilized DGEA. Post-treatment with M1 media, conditioned media, or cell supernatant was collected. The concentrations of TNFα were measured by utilizing a mouse ELISA kit (Ray-Biotech) and following manufacturer's instructions. Student's t-test was used to determine differences in cytokine expression between the treatment groups.

### Statistical Analyses

Throughout the experiments, i.e. soluble DGEA delivery in a 2D environment on TCP, soluble DGEA delivery in a 3D matrix, as well as immobilized DGEA in a 3D matrix, the total cell count of iNOS⁺ and DAPI⁺ cells was exported into GraphPad Prism 8.4.1 (La Jolla, CA). iNOS⁺ cells were normalized to DAPI⁺ cells. M1 macrophages across all experiments, with and without the presence of DGEA, were paired and an Independent Student's t-test was employed to investigate the difference between means. The number of samples ranged from 4-8 per condition in all of the analyses. Statistical significance is reported as p<0.05. The confidence interval is reported as 95%. Results are presented as the mean with ±standard deviation.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A crosslinked poly(alkylene glycol)-based hydrogel composition, comprising:
a cell adhesive peptide covalently conjugated with a first poly(alkylene glycol);
a cleavable peptide linker covalently conjugated with a second and third poly(alkylene glycol); and
DGEA (SEQ ID NO: 12) covalently conjugated with a fourth poly(alkylene glycol);
wherein, said first, second, third, and fourth poly(alkylene glycol), in each instance, are the same or different.

2. The crosslinked poly(alkylene glycol)-based hydrogel composition of claim 1, wherein said cell adhesive peptide in said cell adhesive peptide covalently conjugated with said first poly(alkylene glycol) is selected from the group consisting of RGDS (SEQ ID NO: 4), RDGS (SEQ ID NO: 5), RGES (SEQ ID NO: 6), REGS (SEQ ID NO: 7), IKVAV (SEQ ID NO: 8), VVIAK (SEQ ID NO: 9), YIGSR (SEQ ID NO: 10), YSRIG (SEQ ID NO: 11), DAEG (SEQ ID NO: 13), and combinations thereof.

3. The crosslinked poly(alkylene glycol)-based hydrogel composition of claim 2, wherein said cell adhesive peptide is RGDS (SEQ ID NO: 4).

4. The crosslinked poly(alkylene glycol)-based hydrogel composition of claim 1, wherein said cleavable peptide linker in said cleavable peptide linker covalently conjugated with a second and third poly(alkylene glycol) is selected from the group consisting of GGGPQGIWGQGK (SEQ ID NO: 1), GGGIQQWGPGGK (SEQ ID NO: 2), and GGGGGIPQQWGK (SEQ ID NO: 3).

5. The crosslinked poly(alkylene glycol)-based hydrogel composition of claim 4, wherein said cleavable peptide linker is GGGPQGIWGQGK (SEQ ID NO: 1).

6. The crosslinked poly(alkylene glycol)-based hydrogel composition of claim 1, wherein said first, second, third, and fourth poly(alkylene glycol), in each instance, is selected from the group consisting of acrylate-PEG-succinimidyl valerate (PEG-SVA), acrylate-PEG-N-hydroxylsuccinimide (PEG-NHS), acrylate-PEG-succinimidyl carboxymethyl ester (PEG-SCM), acrylate-PEG-succinimidyl amido succinate (PEG-SAS), acrylate-PEG-succinimidyl carbonate (PEG-SC), acrylate-PEG-succinimidyl glutarate (PEG-SG), acrylate-PEG-succnimidyl succinate (PEG-SS), and acrylate-PEG-maleimide (PEG-MAL).

7. The crosslinked poly(alkylene glycol)-based hydrogel composition of claim 6, wherein said first, second, third, and fourth poly(alkylene glycol) are acrylate-PEG-succinimidyl valerate (PEG-SVA).

8. The crosslinked poly(alkylene glycol)-based hydrogel composition of claim 1, wherein the conjugate of each of said cell adhesive peptide covalently conjugated with said first poly(alkylene glycol), said cleavable peptide linker covalently conjugated with said second and third poly(alkylene glycol), and said DGEA (SEQ ID NO: 12) covalently conjugated with said fourth poly(alkylene glycol), comprises:
an acrylate-PEG cell adhesive peptide conjugate;
an acrylate-PEG cleavable peptide linker conjugate comprising a first and second acrylate-PEG group, wherein said cleavable peptide linker is disposed between said first and second acrylate-PEG group; and
an acrylate-PEG DGEA conjugate.

9. The crosslinked poly(alkylene glycol)-based hydrogel composition of claim 1, wherein said cell adhesive peptide covalently conjugated with a first poly(alkylene glycol) is present in said crosslinked poly(alkylene glycol)-based hydrogel composition at a concentration of about 0.5 mM to about 10 mM.

10. The crosslinked poly(alkylene glycol)-based hydrogel composition of claim 1, wherein said cell adhesive peptide covalently conjugated with a first poly(alkylene glycol) is present in said crosslinked poly(alkylene glycol)-based hydrogel composition at a concentration of about 3.5 mM.

11. The crosslinked poly(alkylene glycol)-based hydrogel composition of claim 1, wherein said DGEA (SEQ ID NO: 12) covalently conjugated with said fourth poly(alkylene glycol) is present in said crosslinked poly(alkylene glycol)-based hydrogel composition at a concentration of about 1 mM to about 15 mM.

12. The crosslinked poly(alkylene glycol)-based hydrogel composition of claim 11, wherein said DGEA (SEQ ID NO: 12) covalently conjugated with said fourth poly(alkylene glycol) is present in said crosslinked poly(alkylene glycol)-based hydrogel composition at a concentration of about 5 mM.

13. The crosslinked poly(alkylene glycol)-based hydrogel composition of claim 1, wherein said cleavable peptide linker covalently conjugated with a second and third poly(alkylene glycol) is present in said crosslinked poly(alkylene glycol)-based hydrogel at about 2 wt % to about 15 wt %.

14. The crosslinked poly(alkylene glycol)-based hydrogel composition of claim 13, wherein said cleavable peptide linker covalently conjugated with a second and third poly(alkylene glycol) is present in said crosslinked poly(alkylene glycol)-based hydrogel at about 5 wt %.

15. A method of treating a disease or disorder associated with excessive or sustained inflammation in a subject in need thereof, comprising administering to said subject a therapeutically effective amount of a composition comprising DGEA.

16. The method of claim 15, wherein said composition comprising DGEA is a hydrogel composition comprising DGEA and one or more synthetic polymers.

17. (canceled)

18. The method of claim 15, wherein said disease or disorder associated with excessive or sustained inflammation is selected from the group consisting of arthritis, asthma, inflammatory bowel disease, chronic obstructive pulmonary disease (COPD), allergic rhinitis, vasculitis, inflammatory neuropathy, psoriasis, systemic lupus erythematosis (SLE), chronic thyroiditis, Hashimoto's thyroiditis, Addison's disease, polymyalgia rheumatica, Sjögren's syndrome, and Churg-Strauss syndrome.

19. (canceled)

20. A method of inhibiting activation of pro-inflammatory M1 macrophages, comprising contacting one or more macrophages with a composition comprising DGEA, wherein said composition comprising DGEA is a hydrogel composition comprising DGEA and one or more synthetic polymers, wherein said hydrogel composition comprising DGEA and one or more synthetic polymers is the crosslinked poly(alkylene glycol)-based hydrogel composition of claim 1.

21-30. (canceled)

31. A method of inhibiting activation of pro-inflammatory M1 macrophages in a subject in need thereof, comprising administering to the subject a therapeutically effective amount of a composition comprising DGEA.

32. The method of claim 31, wherein said composition comprising DGEA is a hydrogel composition comprising DGEA and one or more synthetic polymers.

33-35. (canceled)
